(19) United States
(12) Patent Application Publication
(10) Pub. No.: US 2015/0111239 A1
(43) Pub. Date: Apr. 23, 2015

(54) MICROFLUIDIC DEVICE AND RELATED METHODS

(71) Applicants: University of Maine System Board of Trustees, Bangor, ME (US); Maine Institute for Human Genetics and Health, Inc, Brewer, ME (US)

(72) Inventors: Scott D. Collins, Bangor, ME (US); Rosemary L. Smith, Bangor, ME (US); Janet M. Hock, Bangor, ME (US)

(21) Appl. No.: 14/312,035

(22) Filed: Jun. 23, 2014

Related U.S. Application Data
(63) Continuation of application No. 13/254,981, filed on Jan. 4, 2012, now abandoned.
(60) Provisional application No. 61/157,439, filed on Mar. 4, 2009.

(51) Int. Cl. C12M 1/34; B01L 3/00; C12M 1/00; G01N 33/50; C12M 3/06

(52) U.S. Cl. CPC C12M 41/46; G01N 33/5008; C12M 41/32; C12M 23/16; C12M 29/10; B01L 3/502776; B01L 2300/0867; B01L 2300/0829; B01L 2400/084; B01L 2200/0694; G01N 2500/10
USPC 435/29; 435/289.1; 29/428

(57) ABSTRACT

A combinatorial microenvironment generator is configured for the generation of arbitrary, user-defined, steady-state, concentration gradients with negligible to no flow through the growth medium to perturb diffusion gradients or cellular growth. More importantly, the absolute concentrations and/or gradients can be dynamically altered upon request both spatially and temporally to impose tailored concentration fields for in-situ stimulus studies. Here, diffusion occurs via an array of ports, each of which can be an independently controlled source/sink. Together, the array of ports establishes a user-defined, 3D concentration profile. Useful methods related to this device are also provided.

### CROSS-REFERENCE TO RELATED APPLICATIONS

This application claims the benefit of U.S. Provisional Application No. 61/157,439 filed Mar. 4, 2009, the disclosure of which is incorporated herein by reference.

### STATEMENT OF FEDERAL SPONSORSHIP

This invention was not funded by the United States government.

### FIELD OF THE INVENTION

The present invention pertains generally to the field of biology, chemistry and physics. This invention pertains to a microfluidic device and methods which can produce user-defined concentrations and/or concentration gradients within an in vitro medium with temporal and spatial control. The resulting microenvironment can produce complex conditions without the presence of potentially perturbating fluid flow. The instrument is capable of indefinitely maintaining concentration profiles, which, in turn provides a variety of opportunities for use.

### BACKGROUND OF THE INVENTION

In many biological and physical systems, information is encoded through gradients including: pharmacokinetic drug dispersions/delivery (Fallon, 2009; Lou 2010), electrophoresis, DNA hybridization kinetics (Schoen 2009), as well as the myriad organism responses to gradients such as those present in the environment, such as pheromones for mating behavior or nutrients for bacterial chemotaxis, and also in vivo electrical, thermal, and chemical gradients such as the milieu of electrochemical signals in the developing brain that guide neuronal axons to establish the functional circuitry of the central nervous system (January, 2003). Although the importance of gradients in biology and physics is well known, how cells respond to them is, unfortunately, not as well known due primarily to the overwhelming challenge to quantify or even detect gradients in vivo. To address these issues, researchers have turned to in vitro systems to more quantitatively define concentrations and gradients. However, simulating complex, multicomponent, and dynamic gradients in vitro still remains difficult.

Over the past decade, a number of experimental platforms have been developed to generate concentration gradients of chemotropic molecules for the study of cellular chemotaxis (Abhyankar, 2006), axon outgrowth (Winter, 2002), and cellular maintenance. An excellent review of methodologies for the generation of concentration gradients is provided by Keenan and Folch (Keenan, 2008). One simple means to study cellular taxis and axon growth in concentration gradients has been to use source/sink configurations where the ends of a capillary tube or microfluidic channel (Lin, 2004) are bathed in different reagent concentrations. Time-invariant, linear, steady-state concentration profiles are achieved according to the source/sink boundary conditions. The results have been encouraging, but the technique is cumbersome to implement and extremely limited in application.

Others have printed adjacent lines of gels containing different concentrations of reagents on substrates and allowed them to “blend” together to create a smooth gradient (Rosoff, 2004). Unfortunately, this and similar techniques produce gradients that degrade with time making them generally unsuited to the long term studies mandated by axon growth, cellular development, or cellular taxis.

Microfluidic networks (Chung, 2005 and Keenan, 2006) have been used to generate arbitrarily shaped gradients (Dertinger, 2001; Jeon, 2002; Weibel, 2006; Kim, 2007, Hattori, 2009) by laminating flow streams of different reagent concentrations. Stable, steady-state gradients are readily achieved with these microsystems, and they have produced significant advances in bioengineering and the general understanding of cellular behavior. However, a serious drawback for these microfluidic gradients is that they require perturbing flows—something usually not experienced in vivo outside of the bloodstream and known to deleteriously affect the behavior of many cells, particularly neural growth (Walker, 2005) and cellular taxis.

An interesting twist in creating stable, non-diffusing concentration gradients has been to bind mediators to a substrate surface or polymer backbone (Smith, 2004; Ranieri, 1993) with varying spatially concentrations of bound mediators. Cells then experience a spatially varying mediator concentration without the cumbersome effects of a constantly degrading diffusion gradient and/or perturbing flows. Although immobilization techniques have proven quite useful, they, unfortunately, lack a certain biological reality. Chemical recognition of bound ligands to surface or polymer always begs the question of activity, interference, and steric hindrance which includes the prevention of cellular uptake. More importantly, bound mediators are fixed and, once cast, allow neither the dynamic temporal nor the dynamic spatial control over concentrations that occurs naturally in living organisms.

Haessler, et al (Haessler, 2009) and Kim, et al (Kim, 2009) described a device in which a microchannel flow is used to create a source/sink diffusion configuration across a membrane.

It is a significant technical challenge to study cellular responses to in vivo gradients quantitatively, which requires knowledge of the actual spatial and temporal concentrations of chemical cues. Although it is well accepted that cells respond to chemical gradients, how they respond is not well understood. For example, in some cases the absolute concentration may illicit a cellular response, while in others, it may be the steepness of the gradient, without regard to concentration. To address these issues, researchers have turned to in vitro systems, but simulating the complex in vivo environment, with multi-component or dynamic gradients remains difficult. Moreover, although it is well accepted that exposure to environmental toxins, both natural and artificially imposed, will predispose a person to disease, the specifics such as dosage time, concentration, and most importantly what specific combination of toxins are required to induce a specific pathology remain unknown. Individual gene variations may also dictate susceptibility to toxin exposures, thereby adding additional complexity. Knowledge of how genetic variants and environmental exposures contribute to disease and normal tissue repair can be effectively used to develop new criteria for earlier diagnosis, and lead to new, more effective and targeted therapies for returning troops. Further, the use of pesticides and herbicides in forestry and the pulp and paper industry, combined with naturally occurring high rates of arsenic and radon, may have increased the risk for cancer in certain geographical areas. For instance, the incidence rate for all cancers for men and women in Maine was the highest in the US a decade ago (US Cancer Registries 2004 data). Early detection is a universal challenge to disease treatment that is particularly relevant to people living in rural communities who are more likely to have long term toxin exposure, yet are less likely to obtain routine medial screenings.

A combinatorial strategy and high throughput technology which is applicable to the study of cells, particularly human cells, in vitro is needed to perform a cost and time effective correlation study to assess the complex interdependency of multiply relevant toxins, pollutants and/or bioagents.

### SUMMARY OF THE INVENTION

The present invention provides a microdevice for the generation of arbitrary, user-defined, steady-state, concentration gradients with negligible to no flow through the growth medium to perturb diffusion gradients or cellular growth. More importantly, the absolute concentrations and/or gradients can be dynamically altered upon request both spatially and temporally to impose tailored concentration fields for in-situ stimulus studies. Here, diffusion occurs via an array of ports, each of which can be an independently controlled source/sink. Together, the array of ports establishes a user-defined, 3D concentration profile.

In one embodiment, the present invention provides a microfluidic device, comprising: at least fluid diffusion ports; at least one cell culture chamber; and at least one means for relaying through the ports to the chamber, wherein the ports open to the chamber. Preferred is such a device which further comprises means to relay waste from the chamber. Also preferred is a device which comprises means for maintaining a fixed concentration at the ports by enabling flow in the channels. Also preferred is such a device which comprises from 2 to 100 diffusion ports, more preferably which comprises 4 to 32 diffusion ports, most preferably which comprises 8 to 16 diffusion ports, most preferably 16 diffusion ports. Also preferred is such a device which comprises 1 to 80 cell culture chambers, more preferably which comprises 1 to 4 cell culture chambers, most preferably which comprises one cell culture chamber. Also preferred is such a device, which further comprises cell culture medium in the cell culture chamber, more preferably which further comprises cells in the cell culture medium. Also preferred is such a device wherein the aperture of the diffusion ports are smaller than 130 μm, preferably less than from about 80 μm to about 100 μm, more preferably from about 60 μm to about 80 μm, more preferably less than 50 μm, most preferably smaller than any cells or other particles within the culture medium.

Also preferred is such a device wherein the cell culture medium comprises a polymer and/or a gel, more preferably, such a device comprises a gel ingredient selected from the group consisting of: Matrigel®; agaropectin; agarose; agar; acrylamide; polyacrylamide; silica gel; sol-gel; aerogel; aquamid; hydrogel; organogel; xerogel; carageenan or wherein the gel comprises an ingredient selected from the group consisting of: nucleic acid; amino acid; carbohydrate; co-factor; mineral; growth factor; chemical; and buffer. Also preferred is such a device wherein the cells are present in a cell culture, and the cell culture comprises cells selected from the group consisting of: neuroblasts; neurons; fibroblasts; myoblasts; myotubes; chondroblasts; chondrocytes; osteoblasts; osteocytes; cardiocytes; smooth muscle cells; epithelial cells; keratinocytes; kidney cells; liver cells; lymphocytes; granulocytes; and macrophages.

In another broad embodiment, there is provided a microfluidic device, comprising: a first layer comprising a planar, rigid base; a second solid layer comprising at least one waste channel, wherein the second layer overlays the first layer; a third solid layer comprising at least one fluidic microchannel and at least two waste vias, wherein the third solid layer overlays the second layer such that the waste vias open to a waste channel; a fourth solid layer comprising at least two diffusion ports and at least one culture chamber, wherein the fourth layer overlays the third layer such that a diffusion port opens to a fluidic microchannel and/or a waste via, and a culture chamber. Preferred is such a device which further comprises a fifth planar, solid layer, wherein the fifth layer overlays the fourth layer. Also preferred is such a device wherein the aperture of the diffusion ports are less than 130 μm, preferably from about 80 μm to about 100 μm, more preferred is such a device wherein the aperture of the diffusion port is preferably from about 60 μm to about 80 μm, more preferred is such a device wherein the aperture of the diffusion port is less than 50 μm, most preferably smaller than any cells or other particles within the culture medium. Also preferred is such a device wherein the second layer comprises a material selected from the group consisting of: silicon; glass; polymeric film; silicone elastomer; photoresist; SU-8; hydrogel; and thermoplastic. Also preferred is such a device wherein the first layer and fifth layer comprises glass and/or wherein the second layer comprises polydimethylsiloxane and/or which further comprises cell culture medium in the cell culture medium chamber. More preferred are those devices wherein the cell culture medium comprises a polymer and/or a gel. Most preferred are those devices wherein the gel comprises an ingredient selected from the group consisting of: Matrigel®; agaropectin; agarose; agar; acrylamide; polyacrylamide; silica gel; sol-gel; aerogel; aquamid; hydrogel; organogel; xerogel; carageenan and/or wherein the gel comprises an ingredient selected from the group consisting of: nucleic acid; amino acid; carbohydrate; co-factor; mineral; growth factor; chemical; and buffer. More preferred are those devices which further comprises cells in the cell culture medium, most preferred are those devices wherein the cells are selected from the group consisting of: neuroblasts; neurons; fibroblasts; myoblasts; myotubes; chondroblasts; chondrocytes; osteoblasts; osteocytes; cardiocytes; smooth muscle cells; epithelial cells; keratinocytes; kidney cells; liver cells; lymphocytes; granulocytes; and macrophages.

In other broad embodiments, methods for using the devices herein are provided.

Provided are methods to identify compositions capable of affecting a cell culture, comprising: introducing at least one fluid comprising a test composition to the diffusion ports of a device herein which comprises cells in the cell chamber; and identifying those compositions capable of affecting a cell culture.

Provided are methods to identify compositions useful to treat infection, comprising: introducing at least one fluid comprising a test composition to the diffusion ports of a device herein which comprises cells in the cell chamber, wherein the cells are infectious cells; and identifying those compositions capable of altering the infectious cells.

Provided are methods to identify compositions capable of affecting at least one disease state, comprising: providing at least one disease state model cell culture to the cell culture chamber of a device herein, introducing at least one fluid comprising a test composition to the diffusion ports of the device; and identifying those compositions capable of affecting the disease state model cell culture.

Provided are methods to identify potential environmental toxins, comprising: providing a cell culture to the cell culture chamber of a device herein, introducing at least one fluid comprising at least one test toxin to the diffusion ports of the device; and identifying those toxins which alter the cell culture as potential environmental toxins.

Provided are methods to monitor the effects of exposure to a test environment, comprising: providing a cell culture to the cell culture chamber of a device herein, introducing at least one fluid comprising at least one environmental test sample to the diffusion ports of the device; and identifying the affects of the environmental test sample on the cell culture as indicative of the effects of exposure to a test environment. Preferred are those methods which are repeated over time.

Provided are methods to diagnose at least one disease state, comprising: providing a device herein, which further comprises a cell culture medium containing at least one test cell sample, introducing at least one fluid comprising at least one composition to the diffusion ports of the device, wherein the composition is capable of providing identification of disease state when diffused in a culture medium containing diseased cells; and identifying whether any disease state is present in the sample.

Provided are methods to assess the prognosis of a patient having a disease, comprising: providing a device herein, which further comprises a cell culture medium containing at least one test cell sample, introducing at least one fluid comprising at least one composition to the diffusion ports of the device, wherein the composition is capable of providing identification of prognosis of disease state when diffused in a culture medium containing diseased cells; and identifying the prognosis of the disease state.

Provided are methods to screen for pathogens in a test sample, comprising: providing a device herein, comprising at least one composition known to interact with a pathogen, introducing at least one fluid comprising at least one test sample to the diffusion ports of the device; and identifying any composition-sample interactions, wherein interactions indicate the presence of a pathogen in the test sample.

Provided are methods to screen chemical libraries for useful substances, comprising: introducing at least one fluid comprising at least one chemical library member to the diffusion ports of a device herein which comprises cells in the cell chamber, and identifying a chemical library member which affects the cell culture as a useful substance.

Also provided are methods to identify pesticides, comprising: introducing at least one fluid comprising at least one test composition to the diffusion ports of a device herein which comprises cells in the cell chamber, wherein the cells are insect cells, and identifying a composition which impairs at least one insect cell as a pesticide. Preferred are those methods wherein the cell culture comprises a cell from a pest affecting a crop selected from the group consisting of: cotton; soybean; corn; potato; peanut; sunflower; canola; olive; alfalfa; oats; wheat; millet; tobacco; sugarcane; sugar beet; trees; bushes; flowers; banana; beans; broccoli; brussel sprouts; cabbage; carrot; cassava; cauliflower; chili; cole crops; cruciferous crops; cucumber; cucurbit crops; eggplant, garlic; leeks; lettuce; citrus; melon; onion; papaya; pepper; solanaceous crops; squash; sweet potato; and tomato.

Provided are methods to identify fungicides, comprising: introducing at least one fluid comprising at least one test composition to the diffusion ports of a device herein which comprises cells in the cell chamber, wherein the cells are fungal cells, and identifying a composition which impairs at least one fungal cell as a fungicide. Preferred are those methods wherein the cell culture comprises a cell from a fungus selected from the group consisting of: yeast; mold; mildew; mushroom; and slime mold.

Provided are methods to identify herbicides, comprising: introducing at least one fluid comprising at least one test composition to the diffusion ports of a device herein which comprises cells in the cell chamber, wherein the cells are plant cells, and identifying a composition which impairs at least one plant cell as a herbicide. Preferred are those methods wherein the cell culture comprises a plant cell of a plant selected from the group consisting of: cotton; soybean; corn; potato; peanut; sunflower; canola; olive; alfalfa; oats; wheat; millet; tobacco; sugarcane; sugar beet; trees; bushes; flowers; banana; beans; broccoli; brussel sprouts; cabbage; carrot; cassava; cauliflower; chili; cole crops; cruciferous crops; cucumber; cucurbit crops; eggplant, garlic; leeks; lettuce; citrus; melon; onion; papaya; pepper; solanaceous crops; squash; sweet potato; and tomato.

Provided are methods to identify rodenticides, comprising: introducing at least one fluid comprising at least one test composition to the diffusion ports of a device herein which comprises cells in the cell chamber, wherein the cells are rodent cells; and identifying a composition which impairs at least one rodent cell as a rodenticide. Preferred are those methods wherein the cell culture comprises a rodent cell from a rodent selected from the group consisting of: mouse; mole; vole; rat; prairie dog; groundhog; and rabbit.

Provided are methods to tailor patient treatment, comprising: introducing at least one fluid comprising at least one test composition to the diffusion ports of a device herein which comprises cells in the cell chamber, wherein the cells are cells obtained from a patient; and indentifying whether the test composition favorably affects the patient's cells. “Favorably” in this contexts includes cell death, impairment, growth, proliferation, changes, or any other outcome that would benefit the patient.

Provided are methods to identify compositions useful to increasing cell growth, comprising: introducing at least one fluid comprising a test composition to the diffusion ports of a device herein which comprises cells in the cell chamber; and identifying those compositions capable of increasing cell growth. Preferred are those methods wherein cell growth increase indicates that the compositions are useful to treat a degenerative disease. More preferred are those methods wherein the degenerative disease is selected from the group consisting of: Alzheimer's, Parkinson's, multiple sclerosis; diabetes type I; stroke; and ischemia.

Provided are methods to identify compositions useful for decreasing cell growth, comprising: introducing at least one fluid comprising a test composition to the diffusion ports of a device herein which comprises cells in the cell chamber; and identifying those compositions capable of decreasing cell growth. Preferred are those methods wherein cell growth decrease indicates that the compositions are useful to treat a proliferation disease. More preferred are those methods wherein the proliferation disease is selected from the group consisting of: cancer; viral tumors; bacterial infection; fungal infection; and sepsis.

Provided are methods to identify compositions useful to increase cell proliferation, comprising: introducing at least one fluid comprising a test composition to the diffusion ports of a device herein which comprises cells in the cell chamber; and identifying those compositions capable of increase cell proliferation. Preferred are those methods wherein cell growth reduction indicates that the compositions are useful to treat a degenerative disease. More preferred are those methods wherein the degenerative disease is selected from the group consisting of: Alzheimer's, Parkinson's, multiple sclerosis; diabetes type I; stroke; and ischemia.

Provided are methods to identify compositions useful to decreasing cell proliferation, comprising: introducing at least one fluid comprising a test composition to the diffusion ports of a device herein which comprises cells in the cell chamber; and identifying those compositions capable of decreasing cell proliferation. Preferred are those methods wherein cell proliferation decrease indicates that the compositions are useful to treat a proliferation disease. Most preferred are those methods wherein the proliferation disease is selected from the group consisting of: cancer; viral tumors; bacterial infection; fungal infection; and sepsis.

Provided are methods to assess the health risk of a population: introducing at least one fluid comprising at least one test sample to the diffusion ports of a device herein which comprises cells in the cell chamber; and identifying whether the test sample alters the cell culture, wherein alteration indicates the health risk of the population. Preferred are those methods which is repeated using samples collected at different times and/or which is repeated using samples collected at different locations and/or which is repeated using at least two populations and/or which further comprises comparing health risk from one sample with health risk from another sample. Preferred are those methods wherein the population is selected from a group consisting of: animals; plants; bacteria; and fungi. Also preferred are those methods wherein the population has been exposed to a circumstance selected from the group consisting of: industrial waste spill; industrial waste exposure; Superfund site exposure; hurricane; flood; earthquake; epidemic; fire; and volcano eruption.

Provided are methods for genetic counseling a patient, comprising: providing a device herein, which further comprises a cell culture medium containing at least one test cell sample; introducing at least one fluid comprising at least one composition to the diffusion ports of the device, wherein the composition is capable of providing identification of genetic propensity for at least one genetic marker when diffused in a culture medium containing test cells; and identifying the genetic propensity for the genetic markers identified in the test sample. Preferred are those methods wherein the cell sample is selected from the group consisting of: the patient's cells; a relative's cells; and a potential donor's cells. Also preferred are those methods which further comprise a step of communicating the genetic propensity for the genetic markers to the patient.

Preferred are any methods wherein the composition is introduced via a manner selected from the group consisting of: regular interval pulses; random pulses; timed pulses; steady flow; pulsed according to a simulated physiologic process; flushed with fluid containing no composition; flushed with fluid containing an additional composition; flushed with identifying markers; and in combination with additional compositions.

Preferred are any methods wherein the test or other composition is selected from the group consisting of: one or more pharmaceutical candidates; one or more environmental compounds; one or more toxins; one or more growth factors; one or more antibodies; one or more nucleic acids; one or more proteins; one or more carbohydrates; one or more bacteria; one or more fungi; one or more eukaryotic cells; one or more chemical compounds; one or more biologic compound; one or more ions; one or more precursors; one or more chromatophore; one or more hormones; and a combination of test compositions.

Preferred are any methods wherein identification is accomplished via an affect selected from the group consisting of: altering proteomic profile; altering morphology; altering number; altering size; altering population distribution; altering nucleic acid profile; altering protein profile; altering signaling profile; altering pH; altering color; altering luminescence; altering radiography; altering solubility; altering viscosity; altering gene expression; altering permeability; and altering diffusion.

Preferred are any methods wherein identification is selected from the group consisting of: microscopic inspection; luminescence; radioactivity; antibody interaction; nucleic acid interaction; protein interaction; binding assay; chromatography; filtration; and PCR.

Preferred are any embodiments wherein the cell culture comprises a cell selected from the group consisting of: neuroblasts; neurons; fibroblasts; myoblasts; myotubes; chondroblasts; chondrocytes; osteoblasts; osteocytes; cardiocytes; smooth muscle cells; epithelial cells; keratinocytes; kidney cells; liver cells; lymphocytes; granulocytes; and macrophages.

Various aspects of this invention will become apparent to those skilled in the art from the following detailed description of the preferred embodiment, when read in light of the accompanying figures.

### BRIEF DESCRIPTION OF THE FIGURES

FIG. 1. is a plan view of a combinatorial microenvironment generator having four access ports in contact with a culture chamber, and supplied by four independently controlled supply channels.

FIG. 2 is perspective view of a culture chamber having a 3×3 array of access ports.

FIG. 3 is a perspective, exploded view, partially cut away, of a combinatorial microenvironment generator having a 2×4 array of access ports, 8 fluid delivery microchannels and one waste channel.

FIG. 4A-B show plan views of a combinatorial microenvironment generator with a 4×4 array of access ports and the associated supply channels. FIG. 4A depicts an exemplary embodiment with a 4×4 array of 20 μm×20 μm diffusion ports, and FIG. 4B depicts an alternative embodiment with a reduced number of I/O connections.

FIG. 5 is a computer generated plot of a normalized, steady-state diffusion profile indicating the diffusion into the culture chamber of a single species fed from an access port at the center (source), surrounded by access ports kept at zero concentration (sinks).

FIGS. 6(a)-6(h) illustrate dynamic creations of arbitrary 2D diffusion profiles as the concentrations at each access port are changed over time.

FIG. 7 is a concentration profile in the xz plane for the computer simulation shown in FIG. 5. The xz slice is taken along the peak concentration of FIG. 5, i.e. C(x,50,z, t=∞).

FIG. 8 is a plot of the concentration at C(50,50, T, t=∞) versus the ratio of the thickness, T, of the culture medium and the diffusion port separation, Δl.

FIG. 9A-B show schematic representations of the device shown in FIG. 4B. FIG. 9A shows the device of FIG. 4B with a different color dye loaded into each microfluidic channel (100) before diffusion profiles have become established. FIG. 9B shows the same set of channels after the dyes were allowed to diffuse for 10 minutes. Specifically, FIG. 9B shows the diffusion of each dye into zones around each diffusion port.

FIG. 10 is a time sequence showing the developing diffusion profiles of fluoroscein conjugated BSA across a 500 mm source/sink configuration in an essentially 1D configuration of the set-up depicted in FIG. 9. Graphs are offset in the vertical direction to more clearly show diffusion profiles. For t=30 min and 60 min, the calculated profiles show semi-infinite diffusion, and a best fit curve to Eq 3 is superimposed on the two graphs, Dᵢ=3×10⁻⁸ cm²/s. The graph at t=180 min shows the transition to linear, steady-state behavior, t=300 min. Linear profiles are superimposed on both graphs for comparison.

FIG. 11 is a schematic diagram showing the general fabrication process flow and assembly of a microfluidic device.

### DETAILED DESCRIPTION OF THE INVENTION

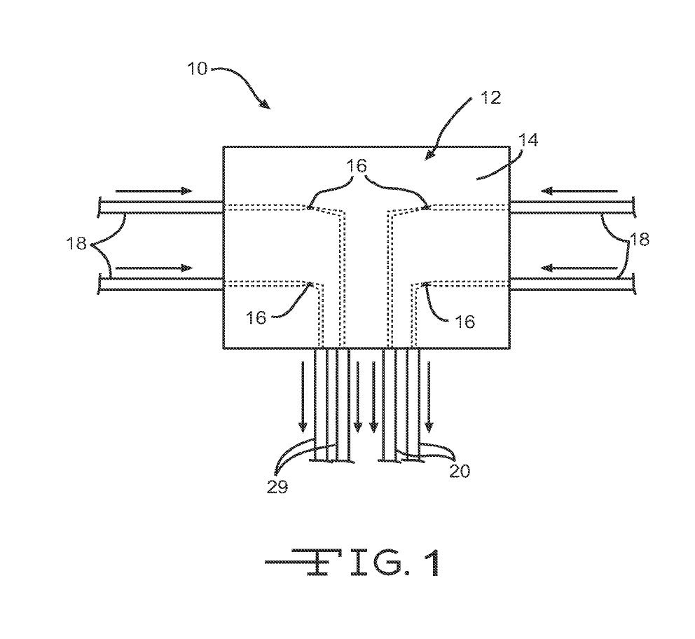

Shown in FIG. 1 is an example of a combinatorial microenvironment generator or microfluidic device 10. The microfluidic device 10 includes a culture chamber 12 for containing the growth medium, not shown in FIG. 1. The growth medium can be any medium suitable for indicating diffusion of the relevant reagents employed in the microfluidic device, including, for example, agarose, Matrigel®, or the like. The floor 14 of the culture chamber 14 includes a 2×2 array of diffusion ports 16. Four microfluidic flow channels 18 are provided to supply fluid containing relevant reagents, such as, for example, ligands, pH, oxygen, mediators, metabolites, and other appropriate reagents. The flow channels 18 can be supplied with the fluid containing the reagent from a source, not shown. The flow rate in each of the flow channels 18 can be controlled by pumps, not shown, or any other suitable means. The concentration of the reagent in the fluid can be maintained constant, or changed over time.

The diffusion ports 16 allow diffusional transfer into the culture chamber 14 while restricting hydrodynamic flow. Steady-state diffusion fields are then established throughout the culture chamber 14 according to the specific source/sink assembly of the diffusion port array. Downstream from the diffusion ports 16 are waste channels 20 for carrying away the fluid in the channels. The waste channels 20 can be combined for mechanical efficiency, as will be described in more detail below.

Within the constraints of diffusion, which are specified in part by the spatial distribution of the diffusion ports, virtually any desired steady-state concentration profile is possible for any number of independently diffusing reagents simply by adjusting the reagent cocktails within each microfluidic flow channel. Additionally, the concentration profiles can be dynamically changed upon user request simply by changing the reagent concentrations within the microfluidic channels. Spatially or temporally oscillating gradients, multiple and/or opposing gradients, as well as more complex gradients can all be readily established. All gradients are steady-state and can be maintained indefinitely as long as reagent flow within the microchannels is maintained to remove/replace diffusional gains/losses at the diffusion ports. This diffusion scenario essentially mimics the biological condition, where chemical mediators generally originate at specific locations and are consumed at equally specific receptor sites.

The diffusional microsystem provides a simple, yet powerful, means to perform quality, long term, in vitro studies in a biologically apropos environment. Computer simulations of diffusion fields and experimental validation of concentration profiles using organic and fluorescent dyes can be used to present an initial characterization of the microfluidic device 10.

A very low flow rate (typically <10 nL/hr) in the underlying micro-channels is sufficient to maintain concentrations at the access ports constant by replenishing/removing any diffusional losses to/from the culture chamber. The concentration profile of bioreagents in the culture matrix are then free to develop under the constraints of diffusion to yield a unique concentration landscape to which the growing cells then respond. Arbitrary steady-state diffusional profiles can be generated by piecewise addition of diffusion profiles between the access ports. This diffusion scenario is essentially identical to that experienced in vivo and the response of cells can be easily monitored and analyzed by microscopy/spectroscopy. By controlling the concentrations flowing in each microchannel, the diffusion profiles in the matrix can be dynamically controlled during cell growth and/or taxis. The transient and steady-state concentration profiles are governed by diffusion for each species present according to Fick's first and second laws. The resultant profiles can be readily simulated given the diffusion coefficients of each introduced species.

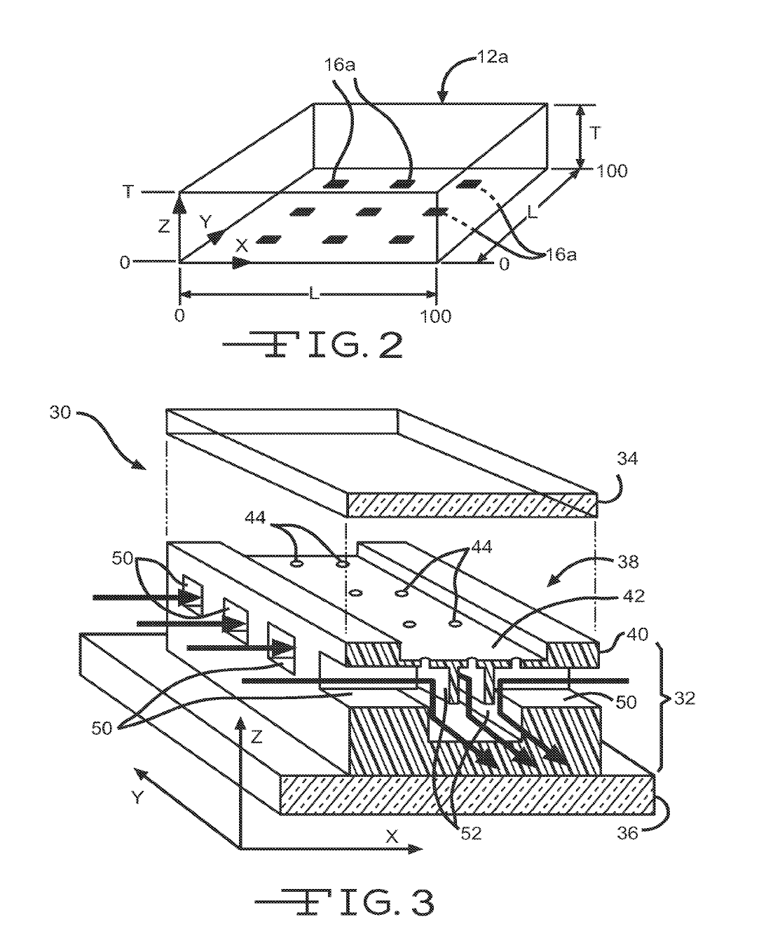

In FIG. 2, a 3×3, 2D array of diffusion ports 16a is shown in the bottom 14a of a culture chamber 12a. It is to be understood that the microfluidic device 10 may contain any number of diffusion ports in any desired geometric configuration.

The present microfluidic device system is a unique and powerful instrument that enables the determination of proteomic profiles from human cell lines after exposure to different combinations of environmental toxins, e.g. arsenic, radon and dioxin, at different dose exposures for varying times. In a manner analogous to how high density interconnections are achieved in integrated circuits, microfabrication technology is employed to create tiny fluidic channels to deliver a programmed combination of environmental pollutants to cell cultures. Cells can be monitored both during and after exposure using optical and mass spectrometry developed for nanovolume analyses.

Specific combinations of pollutants and/or toxins will trigger selective and specific changes in the proteome profile. Within this altered proteome, pathogenomic markers will be identified as predictive of induced cell damage, death or oncogenesis. Cancer tissue samples from humans can then be screened for these candidate pathogenomonic markers. The data can be correlated with information in databases, including those with information pertaining to environmental pollutant exposures, history of military service, genealogy, and demographic data.

The present microfluidic technology allows tracking of the epidemiology of diseases caused by environmental toxins and has a profound impact on our understanding, detection and methods of treatment. Additionally, the combinatorial nature of the proposed instrument has applications to other arenas of biomedical research, including assessing synergies and cross-reactivity of multiple drugs and optimizing tissue engineering, and has the potential to be a transformative technology which could reduce the exorbitant costs of pharmaceutical R&D.

Knowledge of how genetic variants and environmental exposures contribute to disease and normal tissue repair can be effectively used to develop new criteria for earlier diagnosis, and lead to new, more effective and targeted therapies for returning troops possibly reducing long term health care costs ordinarily borne by Department of Defense or the Veteran's Administration.

Microfluidic Systems.

A microfluidic system generally comprises any system in which very small volumes of fluid are stored and manipulated, generally less than about 500 μL, typically less than about 100 μL, and more typically less than about 10 μL. Microfluidic systems carry fluid in predefined paths through one or more microfluidic flow channels. A microfluidic passage may have a minimum dimension, generally height or width, of less than about 200, 100, or 50 μm. Flow channels are described in more detail below.

Microfluidic systems may include one or more sets of flow channels that interconnect to form a generally closed microfluidic network. Such a microfluidic network may include one, two, or more openings at network termini, or intermediate to the network, that interfaces with the external world. Such openings may receive, store, and/or dispense fluid. Dispensing fluid may be directly into the microfluidic network or to sites external the microfluidic system. Such openings generally function in input and/or output mechanisms, described in more detail below, and may include reservoirs.

Microfluidic systems also may include any other suitable features or mechanisms that contribute to fluid, reagent, and/or particle manipulation or analysis. For example, microfluidic systems may include regulatory or control mechanisms that determine aspects of fluid flow rate and/or path. Valves and/or pumps that may participate in such regulatory mechanisms are described in more detail below. Alternatively, or in addition, microfluidic systems may include mechanisms that determine, regulate, and/or sense fluid temperature, fluid pressure, fluid flow rate, chemical composition, exposure to light, exposure to electric fields, magnetic field strength, and/or the like. Accordingly, microfluidic systems may include heaters, coolers, electrodes, lenses, gratings, light sources, pressure sensors, pressure transducers, microprocessors, microelectronics, and/or so on. Furthermore, each microfluidic system may include one or more features that act as a code to identify a given system. The features may include any detectable shape or symbol, or set of shapes or symbols, such as black-and-white or colored barcode, a word, a number, and/or the like, that has a distinctive position, identity, and/or other property (such as optical property).

Materials.

Microfluidic systems may be formed of any suitable material or combination of suitable materials. Suitable materials may include elastomers, such as polydimethylsiloxane (PDMS); plastics, such as polystyrene, polypropylene, polycarbonate, etc.; glass; SU-8; ceramics; sol-gels; silicon and/or other metalloids; metals or metal oxides; biological polymers, mixtures, and/or particles, such as proteins (gelatin, polylysine, serum albumin, collagen, etc.), nucleic acids, microorganisms, etc.; and/or the like. Other materials are known in the art.

Methods of Fabrication.

Microfluidic systems, also referred to as lab-on-chip, chips, devices or apparati, may be fabricated as a unitary structure from a single component, or as a multi-component structure of two or more components. The two or more components may have any suitable relative spatial relationship and may be attached to one another by any suitable bonding mechanism.

In some embodiments, two or more of the components may be fabricated as relatively thin layers, which may be disposed face-to-face. The relatively thin layers may have distinct thickness, based on function. For example, the thickness of some layers may be about 10 to 250 μm, 20 to 200 μm, or about 50 to 150 μm, among others. Other layers may be substantially thicker, in some cases providing mechanical strength to the system. The thicknesses of such other layers may be about 0.25 to 1 mm, 0.1 to 1.5 cm, or 0.5 to 1 cm, among others. Silicon substrates are typically 250 to 1000 microns thick (0.25 to 1 mm). Glass substrates are typically 500 microns to 2 mm thick, and plastics, like plexiglass, are similar to glass, but have been up to 1 cm thick, in microfluidic applications. One or more additional layers may be a substantially planar layer that functions as a substrate layer, in some cases contributing a floor portion to some or all microfluidic flow channels.

Components of a microfluidic system may be fabricated by any suitable mechanism, based on the desired application for the system and on materials used in fabrication. For example, one or more components may be molded, stamped, and/or embossed using a suitable mold. Such a mold may be formed of any suitable material by micromachining, etching, soft lithography, material deposition, cutting, and/or punching, among others. Alternatively, or in addition, components of a microfluidic system may be fabricated without a mold by etching, micromachining, cutting, punching, and/or material deposition.

Microfluidic components may be fabricated separately, joined, and further modified as appropriate. For example, when fabricated as distinct layers, microfluidic components may be bonded, generally face-to-face. These separate components may be surface-treated, for example, with reactive chemicals to modify surface chemistry, with particle binding agents, with reagents to facilitate analysis, and/or so on. Such surface-treatment may be localized to discrete portions of the surface or may be relatively non-localized. In some embodiments, separate layers may be fabricated and then punched and/or cut to produce additional structure. Such punching and/or cutting may be performed before and/or after distinct components have been joined.

Physical Structures of Fluid Networks.

Microfluidic systems may include any suitable structure(s) for the integrated manipulation of small volumes of fluid, including moving and/or storing fluid, and particles associated therewith, for use in particle assays. The structures may include flow channels, reservoirs, and/or regulators, among others. An example of a slightly more complicated microfluidic device 30 is illustrated in FIG. 3. The microfluidic device 30 is generally made of three layers, a core layer 32 sandwiched between an upper layer 34 and a lower layer 36. The upper and lower layers 34, 36 can be made of any suitable non-reacting substance, such as glass. The core layer 32 includes the culture chamber 38 defined by an upper body portion 40 of the core layer 32 and by the upper layer 34. The floor 42 of the culture chamber 38 includes a 2×4 array of diffusion ports 40. One pair of the diffusion ports 44 is shown as being cut away for purposes of illustration.

Eight different flow channels 50 are positioned to provide 8 different fluid compositions or concentrations to the 8 different diffusion ports 44. Once the fluid in a flow channel 50 reaches its associated diffusion port 44, the fluid then flows out of the microfluidic device 30 via waste channels 52. As shown, the waste channels can be combined into a single waste stream downstream from the diffusion ports 44.

Diffusion Structure and Function.

The present invention provides structure and function not found in other microfluidic devices. In particular, this invention provides a means to generate a multi-dimensional concentration gradient with one or more diffusible species in a fluid as inputs. The species may be the same exact concentration and composition, different concentrations and the same composition, the same concentration of different compositions, or different concentrations and different compositions. When the species are supplied to the diffusion ports via a passage or channel, the species diffuse into the culture medium in a manner that is dependent on a variety of user controls, especially their concentration in the supplying channel. The resulting multi-dimensional gradients produce a readable cell “map” of conditions occurring in the culture medium. This map greatly simplifies combinatorial experiments in that one the gradient presents different, but predictable concentrations of species to different cells within a single chamber, eliminating the need to produce serial dilutions/combinations in a well plate or the like. Indeed, this invention provides for “dialing” of conditions and resolution. In the extreme, the resolution of the present invention could be infinite, in the event that infinite ports were physically possible. In this regard, the user may choose the resolution level based on individual need. As the number of ports increase and the diameter of the ports decrease, the finer the control of the gradients, and the higher the resolution. The concept is similar to resolution of digital images: the more pixels in a digital image, the higher the resolution.

The aperture of the diffusion ports is dependent on user need. However, most users will prefer to use a device herein with diffusion port apertures less than 130 microns (used herein interchangeably with μm and micrometer), simply for the reasons that cells or other particles in the culture medium are unlikely to be larger than that size, and therefore resolution would not be ideal. However, the apertures themselves need not be of identical size. Ideally, diffusion port aperture size is selected based on the size and confluency of the cells or other materials in the culture medium, with the ideal aperture being smaller than the cells so as to prevent clogging the aperture. In this regard, if the smallest cells in the medium are 15 microns and not tightly packed in the culture medium, then the port is, for instance, 17 microns or less, ideally 15 microns or less, more ideally, 12 microns or less. The reliability of the diffusion and the resolution would be higher with the smaller apertures. In the event that the smallest cells in the culture medium are 10 microns, and the cells are confluent, the aperture sizes would ideally be 10 microns or less, more ideally 9 microns or less, most ideally 8 microns or less. The following table indicates some examples of cell size to port aperture size relationships.

| | Large | Medium | Small |
|---|---|---|---|
| | Diffusion Port | Diffusion Port | Diffusion Port |
| Cell diameter | Aperture diameter | diameter | diameter |
| (microns) | (microns) | (microns) | (microns) |
| 100 | 70-110 | 30-70 | Less than 30 |
| 90 | 60-95 | 30-90 | Less than 30 |
| 80 | 50-85 | 30-80 | Less than 30 |
| 70 | 40-75 | 30-70 | Less than 30 |
| 60 | 30-65 | 20-60 | Less than 20 |
| 50 | 25-55 | 20-50 | Less than 20 |
| 40 | 25-45 | 20-40 | Less than 20 |
| 30 | 20-35 | 20-30 | Less than 20 |
| 20 | 15-25 | 15-20 | Less than 15 |
| 15 | 10-18 | 10-15 | Less than 10 |
| 10 | 7-13 | 7-10 | Less than 7 |
| 5 | 4-7 | 4-5 | Less than 5 |

The above table is not meant to be limiting; indeed, the aperture of the diffusion ports may be any size that meets the needs of the user. For instance, some fish eggs are larger than 100 microns, and the present device could be used to diagnose, treat, or otherwise study or manipulate the fish eggs.

The shape of the aperture of the port is not essential, and can be oval, square, rectangular, round, hexagonal, etc.; essentially may be any shape. The user may define the shape according to need. Preferred are square or round port shapes, since those are the most easily manufactured at the micron scale at this time. The table above, while indicating diameter, is informative for any type of shape. The resolution of the gradient and potential for cells clogging the port or factors leaking into the port are the informative parameters.

The culture chamber(s) may be any shape or size, although the benefits of the present invention include the microfluidic scale and multi-dimensional gradient mapping. In that regard, one culture chamber is ideal, but a device with two or more culture chambers is also within the scope of the present invention. Chamber height has a lower limit of the dimension of the cells it contains and an upper limit set by the desired time to reach steady-state concentration profiles. Side length has a lower limit again set by the cell dimension and also by how many ports are needed to establish the desired concentration profile and the smallest size port that can be fabricated.

| Very small chamber | Small chamber | Medium chamber | Large chamber |
|---|---|---|---|
| dimensions | dimensions | dimensions | dimensions |
| 100 μm × 100 | 1 mm × 500 | 2 mm × 2 | 2.5 cm × 2.5 |
| μm × 10 μm | μm × 25 μm | mm × 50 μm | cm × 250 μm |

The flow rates of the fluid to the device may be any that the user prefers, as long as it is sufficient to maintain a constant concentration of diffusing species at the ports, over the time period desired to maintain a concentration profile inside the chamber. Some examples are provided herein.

| Very Low | Low | Medium | High |
|---|---|---|---|
| Flow Rates | Flow Rates | Flow Rates | Flow Rates |
| <Ω1 pL/min | <10 pL/min | 0.01-1 nL/min | >10 nL/min |

The degree of control over a 2D concentration profile that can be generated with the present invention depends on the number, aperture and spacing of the diffusion ports. The following ranges for the number of ports per chamber are useful for many common applications, but are not intended as limitations.

| Low | Medium | High | Very High |
|---|---|---|---|
| Port Number | Port Number | Port Number | Port Number |
| 2-4 | 4-9 | 9-20 | >20 |

Flow Channels.

Flow channels, sometimes referred to as passages, generally comprise any suitable path, channel, or duct through, over, or along which materials (e.g., fluid, particles, and/or reagents) may pass in a microfluidic system. Collectively, a set of fluidically communicating flow channels, generally in the form of channels, may be referred to as a microfluidic network. In some cases, flow channels may be described as having surfaces that form a floor, a roof, and walls. Flow channels may have any suitable dimensions and geometry, including width, height, length, and/or cross-sectional profile, among others, and may follow any suitable path, including linear, circular, and/or curvilinear, among others. Flow channels also may have any suitable surface contours, including recesses, protrusions, and/or apertures, and may have any suitable surface chemistry or permeability at any appropriate position within a channel. Suitable surface chemistry may include surface modification, by addition and/or treatment with a chemical and/or reagent, before, during, and/or after passage formation.

In some cases, the flow channels may be described according to function. For example, flow channels may be described according to direction of material flow in a particular application, relationship to a particular reference structure, and/or type of material carried. Accordingly, flow channels may be inlet flow channels (or channels), which generally carry materials to a site, and outlet flow channels (or channels), which generally carry materials from a site. In addition, flow channels may be referred to as particle flow channels (or channels), reagent flow channels (or channels), focusing flow channels (or channels), perfusion flow channels (or channels), waste flow channels (or channels), and/or the like.

Flow channels may branch, join, and/or dead-end to form any suitable microfluidic network. Accordingly, flow channels may function in particle positioning, sorting, retention, treatment, detection, propagation, storage, mixing, and/or release, among others.

Reservoirs.

Reservoirs generally comprise any suitable receptacle or chamber for storing materials (e.g., fluid, particles and/or reagents), before, during, between, and/or after processing operations (e.g., measurement and/or treatment). Reservoirs, also referred to as wells or waste chambers, may include input, intermediate, and/or output reservoirs. Input reservoirs may store materials (e.g., fluid, particles, and/or reagents) prior to inputting the materials to a microfluidic network(s) portion of a chip. By contrast, intermediate reservoirs may store materials during and/or between processing operations. Finally, output reservoirs may store materials prior to outputting from the chip, for example, to an external processor or waste, or prior to disposal of the chip.

Regulators.

Regulators generally comprise any suitable mechanism for generating and/or regulating movement of materials (e.g., fluid, particles, and/or reagents). Suitable regulators may include valves, pumps, and/or electrodes, among others. Regulators may operate by actively promoting flow and/or by restricting active or passive flow. Suitable functions mediated by regulators may include mixing, sorting, connection (or isolation) of fluidic networks, and/or the like.

Particles.

The microfluidic systems herein may be used to manipulate and/or analyze virtually any particles. A particle generally comprises any object that is small enough to be inputted and manipulated within a microfluidic network in association with fluid, but that is large enough to be distinguishable from the fluid. Particles, as used here, typically are microscopic or near-microscopic, and may have diameters of about 0.005 to 100 μm, 0.1 to 50 μm, or about 0.5 to 30 μm. Alternatively, or in addition, particles may have masses of about 10-20 to 10-5 grams, 10-16 to 10-7 grams, or 10-14 to 10-8 grams. Exemplary particles may include cells, viruses, organelles, beads, and/or vesicles, and aggregates thereof, such as dimers, trimers, etc.

Cells.

Cells, as used here, generally comprise any self-replicating, membrane-bounded biological entity, or any non-replicating, membrane-bounded descendant thereof. Non-replicating descendants may be senescent cells, terminally differentiated cells, cell chimeras, serum-starved cells, infected cells, non-replicating mutants, anucleate cells, stem cells, genetically-modified cells, etc.

Cells used as particles in microfluidic systems may have any suitable origin, genetic background, state of health, state of fixation, membrane permeability, pretreatment, and/or population purity, among others. Origin of cells may be eukaryotic, prokaryotic, archae, etc., and may be from animals, plants, fungi, protists, bacteria, and/or the like. Cells may be wild-type; natural, chemical, or viral mutants; engineered mutants (such as transgenics); and/or the like. In addition, cells may be growing, quiescent, senescent, transformed, and/or immortalized, among others, and cells may be fixed and/or unfixed. Living or dead, fixed or unfixed cells may have intact membranes, and/or permeabilized/disrupted membranes to allow uptake of ions, labels, dyes, ligands, etc., or to allow release of cell contents. Cells may have been pretreated before introduction into a microfluidic system by any suitable processing steps. Such processing steps may include modulator treatment, transfection (including infection, injection, particle bombardment, lipofection, coprecipitate transfection, etc.), processing with assay reagents, such as dyes or labels, and/or so on. Furthermore, cells may be a monoculture, generally derived as a clonal population from a single cell or a small set of very similar cells; may be presorted by any suitable mechanism such as affinity binding, FACS, drug selection, etc.; and/or may be a mixed or heterogeneous population of distinct cell types.

Eukaryotic Cells.

Eukaryotic cells, that is, cells having one or more nuclei, or anucleate derivatives thereof, may be obtained from any suitable source, including primary cells, established cells, and/or patient samples. Such cells may be from any cell type or mixture of cell types, from any developmental stage, and/or from any genetic background. Furthermore, eukaryotic cells may be adherent and/or non-adherent. Such cells may be from any suitable eukaryotic organism including animals, plants, fungi, and/or protists.

Eukaryotics cells may be from animals, that is, vertebrates or invertebrates. Vertebrates may include mammals, that is, primates (such as humans, apes, monkeys, etc.) or nonprimates (such as cows, horses, sheep, pigs, dogs, cats, marsupials, rodents, and/or the like). Nonmammalian vertebrates may include birds, reptiles, fish, (such as trout, salmon, goldfish, zebrafish, etc.), and/or amphibians (such as frogs of the species Xenopus, Rana, etc.). Invertebrates may include arthropods (such as arachnids, insects (e.g., Drosophila), etc.), mollusks (such as clams, snails, etc.), annelids (such as earthworms, etc.), echinoderms (such as various starfish, among others), coelenterates (such as jellyfish, coral, etc.), porifera (sponges), platyhelminths (tapeworms), nemathelminths (flatworms), etc.

Eukaryotic cells may be from any suitable plant, such as monocotyledons, dicotyledons, gymnosperms, angiosperms, ferns, mosses, lichens, and/or algae, among others. Exemplary plants may include plant crops (such as rice, corn, wheat, rye, barley, potatoes, etc.), plants used in research (e.g., Arabadopsis, loblolly pine, etc.), plants of horticultural values (ornamental palms, roses, etc.), and/or the like.

Eukaryotic cells may be from any suitable fungi, including members of the phyla Chytridiomycota, Zygomycota, Ascomycota, Basidiomycota, Deuteromycetes, and/or yeasts. Exemplary fungi may include Saccharomyces cerevisiae, Schizosaccharomyces pombe, Pichia pastoralis, Neurospora crassa, mushrooms, puffballs, imperfect fungi, molds, and/or the like.

Eukaryotic cells may be from any suitable protists (protozoans), including amoebae, ciliates, flagellates, coccidia, microsporidia, and/or the like. Exemplary protists may include Giardia lamblia, Entamoeba, histolytica, Cryptosporidium, and/or N. fowleri, among others.

Particles may include eukaryotic cells that are primary, that is, taken directly from an organism or nature, without subsequent extended culture in vitro. For example, the cells may be obtained from a patient sample, such as whole blood, packed cells, white blood cells, urine, sputum, feces, mucus, spinal fluid, tumors, diseased tissue, bone marrow, lymph, semen, pleural fluid, a prenatal sample, an aspirate, a biopsy, disaggregated tissue, epidermal cells, keratinocytes, endothelial cells, smooth muscle cells, skeletal muscle cells, neural cells, renal cells, prostate cells, liver cells, stem cells, osteoblasts, and/or the like. Similar samples may be manipulated and analyzed from human volunteers, selected members of the human population, forensic samples, animals, plants, and/or natural sources (water, soil, air, etc.), among others.

Alternatively, or in addition, particles may include established eukaryotic cells. Such cells may be immortalized and/or transformed by any suitable treatment, including viral infection, nucleic acid transfection, chemical treatment, extended passage and selection, radiation exposure, and/or the like. Such established cells may include various lineages such as neuroblasts, neurons, fibroblasts, myoblasts, myotubes, chondroblasts, chondrocytes, osteoblasts, osteocytes, cardiocytes, smooth muscle cells, epithelial cells, keratinocytes, kidney cells, liver cells, lymphocytes, granulocytes, and/or macrophages, among others. Exemplary established cell lines may include Rat-1, NIH 3T3, HEK 293, COS1, COS7, CV-1, C2C12, MDCK, PC12, SAOS, HeLa, Schneider cells, Junkat cells, SL2, and/or the like.

Prokaryotic Cells.

Particles may be prokaryotic cells, that is, self-replicating, membrane-bounded microorganisms that lack membrane-bound organelles, or nonreplicating descendants thereof. Prokaryotic cells may be from any phyla, including Aquificae, Bacteroids, Chlorobia, Chrysogenetes, Cyanobacteria, Fibrobacter, Firmicutes, Flavobacteria, Fusobacteria, Proteobacteria, Sphingobacteria, Spirochaetes, Thermomicrobia, and/or Xenobacteria, among others. Such bacteria may be gram-negative, gram-positive, harmful, beneficial, and/or pathogenic. Exemplary prokaryotic cells may include E. coli, S. typhimurium, B. subtilis, S. aureus, C. perfringens, V. parahaemolyticus, and/or B. anthracis, among others.

Culture Conditions.

The cell culture mechanism may culture cells under any suitable environmental conditions using any appropriate environmental control mechanisms. Suitable environmental conditions may include a desired gas composition, temperature, rate and frequency of media exchange, and/or the like. Environmental control mechanisms may operate internal and/or external to a microfluidic system. Internal mechanisms may include on-board heaters, gas conduits, and/or media reservoirs. External mechanisms may include an atmosphere- and/or temperature-controlled incubator/heat source, and/or a media source external to the system. An atmosphere-controlled incubator may be more suitable when the system is at least partially formed of a gas-permeable material, such as PDMS. Media, including gas-conditioned media, may be introduced from an external source by any suitable input mechanism, including manual pipetting, automated pipetting, noncontact spitting, etc. In some embodiments, the chip may be pre-incubated with media, which may then be discarded, prior to the introduction of cells and/or other biological materials.

Viruses.

Viruses may be manipulated and/or analyzed as particles in microfluidic systems. Viruses generally comprise any microscopic/submicroscopic parasites of cells (animals, plants, fungi, protists, and/or bacteria) that include a protein and/or membrane coat and that are unable to replicate without a host cell. Viruses may include DNA viruses, RNA viruses, retroviruses, virions, viroids, prions, etc. Exemplary viruses may include HIV, RSV, rabies, hepatitis virus, Epstein-Barr virus, rhinoviruses, bacteriophages, prions that cause various diseases (CJD (Creutzfeld-Jacob disease, kuru, GSS (Gerstmann-Straussler-Scheinker syndrome), FFI (Fatal Familial Insomnia), Alpers syndrome, etc.), and/or the like.

Organelles.

Organelles may be manipulated and/or analyzed in microfluidic systems. Organelles generally comprise any particulate component of a cell. For example, organelles may include nuclei, Golgi apparatus, lysosomes, endosomes, mitochondria, peroxisomes, endoplasmic reticulum, phagosomes, vacuoles, chloroplasts, etc.

Beads.

Particle assays may be performed with beads. Beads generally comprise any suitable manufactured particles. Beads may be manufactured from inorganic materials, or materials that are synthesized chemically, enzymatically and/or biologically. Furthermore, beads may have any suitable porosity and may be formed as a solid or as a gel. Suitable bead compositions may include plastics (e.g., polystyrene), dextrans, glass, ceramics, sol-gels, elastomers, silicon, metals, and/or biopolymers (proteins, nucleic acids, etc.). Beads may have any suitable particle diameter or range of diameters. Accordingly, beads may be a substantially uniform population with a narrow range of diameters, or beads may be a heterogeneous population with a broad range of diameters, or two or more distinct diameters.

Beads maybe associated with any suitable materials. The materials may include compounds, polymers, complexes, mixtures, phages, viruses, and/or cells, among others. For example, the beads may be associated with a member of a specific binding pair, such as a receptor, a ligand, a nucleic acid, a member of a compound library, and/or so on. Beads may be a mixture of distinct beads, in some cases carrying distinct materials. The distinct beads may differ in any suitable aspect(s), such as size, shape, an associated code, and/or material carried by the beads. In some embodiments, the aspect may identify the associated material. Codes are described further herein.

Vesicles.

Particles may be vesicles. Vesicles generally comprise any non-cellularly derived particle that is defined by a lipid envelope. Vesicles may include any suitable components in their envelope or interior portions. Suitable components may include compounds, polymers, complexes, mixtures, aggregates, and/or particles, among others. Exemplary components may include proteins, peptides, small compounds, drug candidates, receptors, nucleic acids, ligands, and/or the like.

Cell-Based Assays/Methods.

The microfluidic systems disclosed in this specification may be used for any suitable cell assays or methods, including any combinations of cells, cell selection(s) (by selective retention), treatment(s), and/or measurement(s), as described herein.

The cell assays may characterize cells, either with or without addition of a modulator. Cell assays may measure cell genotypes, phenotypes, and/or interactions with modulators. These assays may characterize individual cells and/or cell populations/groups of any suitable size. Cells may be characterized in the absence of an added modulator to define one or more characteristics of the cells themselves. Alternatively, or in addition, cell may be characterized in the presence of an added modulator to measure interaction(s) between the cells and the modulator. Moreover, cells may be exposed to a selected concentration of a reagent, or a plurality of concentrations of a reagent. In other embodiments, cells are exposed to a gradient of concentrations of reagent to determine whether such cells will be attracted or repelled by increasing amounts of such reagent.

In other embodiments, a first type of cell is grown in fluid communication with a second type of cell, wherein the first type of cell is affected by the presence of the second type of cell, preferably as a co-culture or feeder type relationship. The cells of the first type and the cells of the second type are kept separate from each other by a retention mechanism, although fluid, preferably liquid, is permitted to be in joint contact with each type of cell so that sub-cellular or biochemical materials may be exchanged between cell types.

Genotypic Assays.

Genotypic assays may be conducted on cells in microfluidic systems to measure the genetic constitution of cells. The genotypic assays may be conducted on any suitable cell or cell populations, for example, patient samples, prenatal samples (such as embryonic, fetal, chorionic villi, etc.), experimentally manipulated cells (such as transgenic cells), and/or so on. Such genotypic aspects may include copy number (such as duplication, deletion, amplification, and/or the like) and/or structure (such as rearrangement, fusion, number of repeats (such as dinucleotide, triplet repeats, telomeric repeats, etc.), mutation, gene/pseudogene, specific allele, presence/absence/identity/frequency of single nucleotide polymorphisms, integration site, chromosomal/episomal, and/or the like) of a nuclear and/or mitochondrial gene(s), genomic region(s), and/or chromosomal region (s) (such as telomeres, centromeres, repetitive sequences, etc.). Methods for genotypic assays may include nucleic acid hybridization in situ (on intact cells/nuclei) or with DNA released from cells, for example, by lysing the cells. Nucleic acid hybridization with nucleic acids may be carried out with a dye-labeled probe, a probe labeled with a specific binding pair, a stem-loop probe carrying an energy transfer pair (such as a “molecular beacon”), and/or with a probe that is labeled enzymatically after hybridization (such as by primer extension with a polymerase, modification with terminal transferase, etc). Alternatively, or in addition, methods for genotypic assays may include polymerase-mediated amplification of nucleic acids, for example, by thermal cycling (PCR) or by isothermal strand-displacement methods. In some embodiments, genotypic assays may use electrophoresis to assist in analysis of nucleic acids. Related gene-based assays may measure other aspects of gene regions, genes, chromosomal regions, whole chromosomes, or genomes, using similar assay methods, and suitable probes or DNA dyes (such as propidium iodide, Hoechst, etc.). These other aspects may include total DNA content (for example 2N, 4N, 8N, etc., to measure diploid, tetraploid, or polyploid genotypes and/or cell cycle distribution), number or position of specific chromosomes, and/or position of specific genes (such as adjacent the nuclear membrane, another nuclear structure, and so on).

Phenotypic Assays.

Phenotypic assays may be conducted to characterize cells in microfluidic systems, based on genetic makeup and/or environmental influences, such as presence of modulators. These assays may measure any molecular or cellular aspect of whole cells, cellular organelles, and/or endogenous (native) or exogenous (foreign) cell constituents/components.

Aspects of a whole cell or whole cell population may include number, size, density, shape, differentiation state, spreading, motility, translational activity, transcriptional activity, mitotic activity, replicational activity, transformation, status of one or more signaling pathways, presence/absence of processes, intact/lysed, live/dead, frequency/extent of apoptosis or necrosis, presence/absence/efficiency of attachment to a substrate (or to a passage), growth rate, cell cycle distribution, ability to repair DNA, response to heat shock, nature and/or frequency of cell-cell contacts, etc.

Aspects of cell organelles may include number, size, shape, distribution, activity, etc. of a cell's (or cell population's) nuclei, cell-surface membrane, lysosomes, mitochondria, Golgi apparatus, endoplasmic reticulum, peroxisomes, nuclear membrane, endosomes, secretory granules, cytoskeleton, axons, and/or neurites, among others.

Aspects of cell constituents/components may include presence/absence or level, localization, movement, activity, modification, structure, etc. of any nucleic acid(s), polypeptide(s), carbohydrate(s), lipid(s), ion(s), small molecule, hormone, metabolite, and/or a complex(es) thereof, among others. Presence/absence or level may be measured relative to other cells or cell populations, for example, with and without modulator. Localization may be relative to the whole cell or individual cell organelles or components. For example, localization may be cytoplasmic, nuclear, membrane-associated, cell-surface-associated, extracellular, mitochondrial, endosomal, lysosomal, peroxisomal, and/or so on. Exemplary cytoplasmic/nuclear localization may include transcription factors that translocate between these two locations, such as NF-κB, NFAT, steroid receptors, nuclear hormone receptors, and/or STATs, among others. Movement may include intracellular trafficking, such as protein targeting to specific organelles, endocytosis, exocytosis, recycling, etc. Exemplary movements may include endocytosis of cell-surface receptors or associated proteins (such as GPCRs, receptor tyrosine kinases, arrestin, and/or the like), either constitutively or in response to ligand binding. Activity may include functional or optical activity, such as enzyme activity, fluorescence, and/or the like, for example, mediated by kinases, phosphatases, methylases, demethylases, proteases, nucleases, lipases, reporter proteins (for example beta-galactosidase, chloramphenicol acetyltransferase, luciferase, glucuronidase, green fluorescent protein (and related derivatives), etc.), and/or so on. Modification may include the presence/absence, position, and/or level of any suitable covalently attached moiety. Such modifications may include phosphorylation, methylation, ubiquitination, carboxylation, and/or farnesylation, among others. Structure may include primary structure, for example after processing (such as cleavage or ligation), secondary structure or tertiary structure (e.g., conformation), and/or quaternary structure (such as association with partners in, on, or about cells). Methods for measuring modifications and/or structure may include specific binding agents (such as antibodies, etc.), in vivo or in vitro incorporation of labeled reagents, energy transfer measurements (such as FRET), surface plasmon resonance, and/or enzyme fragment complementation or two-hydrid assays, among others.

Nucleic acids may include genomic DNA, mitochondrial DNA, viral DNA, bacterial DNA, phage DNA, synthetic DNA, transfected DNA, reporter gene DNA, etc. Alternatively, or in addition, nucleic acids may include total RNAs, hnRNAs, mRNAs, tRNAs, siRNAs, dsRNAs, snRNAs, ribozymes, structural RNAs, viral RNAs, bacterial RNAs, gene-specific RNAs, reporter RNAs (expressed from reporter genes), and/or the like. Methods for assaying nucleic acids may include any of the techniques listed above under genotypic assays. In addition, methods for assaying nucleic acids may include ribonuclease protection assays.

Polypeptides may include any proteins, peptides, glycoproteins, proteolipids, etc. Exemplary polypeptides include receptors, ligands, enzymes, transcription factors, transcription cofactors, ribosomal components, regulatory proteins, cytoskeletal proteins, structural proteins, channels, transporters, reporter proteins (such as those listed above which are expressed from reporter genes), and/or the like. Methods for measuring polypeptides may include enzymatic assays and/or use of specific binding members (such as antibodies, lectins, etc.), among others. Specific binding members are described herein.

Carbohydrates, lipids, ions, small molecules, and/or hormones may include any compounds, polymers, or complexes. For example, carbohydrates may include simple sugars, di- and polysaccharides, glycolipids, glycoproteins, proteoglycans, etc. Lipids may include cholesterol and/or inositol lipids (e.g., phosphoinositides), among others; ions may include calcium, sodium, chloride, potassium, iron, zinc, hydrogen, magnesium, heavy metals, and/or manganese, among other; small molecules and/or hormones may include metabolites, and/or second messengers (such as cAMP or cGMP, among others), and/or the like. Concentration gradients and/or movement of ions may provide electrical measurements, for example, by patch-clamp analysis, as described herein.

Interaction Assays.

Interaction generally comprises any specific binding of a modulator to a cell or population of cells, or any detectable change in a cell characteristic in response to the modulator. Specific binding is any binding that is predominantly to a given partner(s) that is in, on, or about the cell(s). Specific binding may have a binding coefficient with the given partner of about 10-3 M and lower, with preferred specific binding coefficients of about 10-4 M, 10-6 M, or 10-8 M and lower. Alternatively, interaction may be any change in a phenotypic or genotypic characteristic, as described above, in response to the modulator.

Interaction assays may be performed using any suitable measurement method. For example, the modulator may be labeled, such as with an optically detectable dye, and may be labeled secondarily after interaction with cells. Binding of the dye to the cell or cells thus may be quantified. Alternatively, or in addition, the cell may be treated or otherwise processed to enable measurement of a phenotypic characteristic produced by modulator contact, as detailed herein.

Cells and/or cell populations may be screened with libraries of modulators to identify interacting modulators and/or modulators with desired interaction capabilities, such as a desired phenotypic effect (such as reporter gene response, change in expression level of a native gene/protein, electrophysiological effect, etc.) and/or coefficient of binding. A library generally comprises a set of two or more members (modulators) that share a common characteristic, such as structure or function. Accordingly, a library may include two or more small molecules, two or more nucleic acids, two or more viruses, two or more phages, two or more different types of cells, two or more peptides, and/or two or more proteins, among others.

Signal Transduction Assays.

Microfluidic assays of cells and/or populations may measure activity of signal transduction pathways. The activity may be measured relative to an arbitrary level of activity, relative to other cells and/or populations (see below), and/or as a measure of modulator interaction with cells (see above). Signal transduction pathways generally comprise any flow of information in a cell. In many cases, signal transduction pathways transfer extracellular information, in the form of a ligand(s) or other modulator(s), through the membrane, to produce an intracellular signal. The extracellular information may act, at least partially, by triggering events at or near the membrane by binding to a cell-surface receptor, such as a G Protein-Coupled Receptor (GPCR), a channel-coupled receptor, a receptor tyrosine kinase, a receptor serine/threonine kinase, and/or a receptor phosphatase, among others. These events may include changes in channel activity, receptor clustering, receptor endocytosis, receptor enzyme activity (e.g., kinase activity), and/or second messenger production (e.g., cAMP, cGMP, diacylglcyerol, phosphatidylinositol, etc.). Such events may lead to a cascade of regulatory events, such as phosphorylation/dephosphorylation, complex formation, degradation, and/or so on, which may result, ultimately, in altered gene expression. In other cases, modulators pass through the membrane and directly bind to intracellular receptors, for example with nuclear receptors (such as steroid receptors (GR, ER, PR, MR, etc.), retinoid receptors, retinoid X receptor (RXRs), thyroid hormone receptors, peroxisome proliferation-activating receptors (PPARs), and/or xenobiotic receptors, among others). Micro-environment information assays are an important aspect of the present invention. Therefore, any suitable aspect of this flow of information may be measured to monitor a particular signal transduction pathway.

The activity measured may be based at least partially, on the type of signal transduction pathway being assayed. Accordingly, signal transduction assays may measure ligand binding; receptor internalization; changes in membrane currents; association of receptor with another factor, such as arrestin, a small G-like protein such as rac, or rho, and/or the like; calcium levels; activity of a kinase, such as protein kinase A, protein kinase C, CaM kinase, myosin light chain kinase, cyclin dependent kinases, P13-kinase, etc.; cAMP levels; phosholipase C activity; subcellular distribution of proteins, for example, NF-κB, nuclear receptors, and/or STATs, among others. Alternatively, or in addition, signal transduction assays may measure expression of native target genes and/or foreign reporter genes that report activity of a signal transduction pathway(s). Expression may be measured as absence/presence or level of RNA, protein, metabolite, or enzyme activity, among others, as described above.

Comparison of Cells and/or Cell Populations.

Cell-based assays may be used to compare genotypic, phenotypic, and/or modulator interaction of cells and/or populations of cells. The cells and/or populations may be compared in distinct microfluidic systems or within the same microfluidic system. Comparison in the same microfluidic system may be conducted in parallel using a side-by-side configuration, in parallel at isolated sites.

Single-Cell Assays.

Microfluidic systems may be used to perform single-cell assays, which generally comprise any assays that are preferably or necessarily performed on one cell at a time. Examples of single cell assays include patch-clamp analysis, single-cell PCR, single-cell fluorescence in situ hybridization (FISH), subcellular distribution of a protein, and/or differentiation assays (conversion to distinct cell types). In some cases, single-cell assays may be performed on a retained group of two or more cells, by measuring an individual characteristic of one member of the group. In other cases, single-cell assays may require retention of a single cell, for example, when the cell is lysed before the assay.

Sorting/Selection.

Microfluidic systems may be used to sort or select single cells and/or cell populations. The sorted/selected cells or populations may be selected by stochastic mechanisms, size, density, magnetic properties, cell-surface properties (that is, ability to adhere to a substrate), growth and/or survival capabilities, and/or based on a measured characteristic of the cells or populations (such as response to a ligand, specific phenotype, and/or the like). Cells and/or populations may be sorted more than once during manipulation and/or analysis in a microfluidic system. In particular, heterogeneous populations of cells, such as blood samples or clinical biopsies, partially transfected or differentiated cell populations, disaggregated tissues, natural samples, forensic samples, etc. may be sorted/selected. Additional aspects of cell sorting and suitable cells and cell populations are described herein.

Storage/Maintenance.

Microfluidic systems may perform storage and/or maintenance functions for cells. Accordingly, cells may be introduced into microfluidic systems and cultured for prolonged periods of time, such as longer than one week, one month, three months, and/or one year. Using microfluidic systems for storage and/or maintenance of cells may consume smaller amounts of media and space, and may maintain cells in a more viable state than other storage/maintenance methods. Additional aspects of storing and maintaining cells in microfluidic systems are included herein.

Application to Other Particles, Fluids, Etc.

Microfluidic systems may be used for any suitable virally based, organelle-based, bead-based, and/or vesicle-based assays and/or methods. Moreover, any particle, such as nucleic acid, amino acid, antibody, small molecule, etc. may be placed in the culture chamber in a medium. The medium is not limited to growth medium in these instances, or in the instance of cells in the medium. Moreover, pockets of fluids or gels may also be trapped in the medium, allowing for versatility of this invention across a wide variety of applications. Furthermore, the methods are also applicable to many industries, such as the cosmetic, consumer goods, military and many others.

These assays may measure binding (or effects) of modulators (compounds, mixtures, polymers, biomolecules, cells, etc.) to one or more materials (compounds, polymers, mixtures, cells, etc.) present in/on, or associated with, any of these other particles. Alternatively, or in addition, these assays may measure changes in activity (e.g., enzyme activity), an optical property (e.g., chemiluminescence, fluorescence, or absorbance, among others), and/or a conformational change induced by interaction.

In some embodiments, beads may include detectable codes. Such codes may be imparted by one or more materials having detectable properties, such as optical properties (e.g., spectrum, intensity, and or degree of fluorescence excitation/emission, absorbance, reflectance, refractive index, etc.). The one or more materials may provide nonspatial information or may have discrete spatial positions that contribute to coding aspects of each code. The codes may allow distinct samples, such as cells, compounds, proteins, and/or the like, to be associated with beads having distinct codes. The distinct samples may then be combined, assayed together, and identified by reading the code on each bead. Suitable assays for cell-associated beads may include any of the cell assays described above.

Input Mechanisms.

Microfluidic systems may include one or more input mechanisms that interface with the microfluidic network(s). An input mechanism generally comprises any suitable mechanism for inputting material(s) (e.g., particles, fluid, and/or reagents) to a microfluidic network of a microfluidic chip, including selective (that is, component-by-component) and/or bulk mechanisms.

Internal/External Sources.

The input mechanism may receive material from internal sources, that is, reservoirs that are included in a microfluidic chip, and/or external sources, that is, reservoirs that are separate from, or external to, the chip. Input mechanisms that input materials from internal sources may use any suitable receptacle to store and dispense the materials. Suitable receptacles may include a void formed in the chip. Such voids may be directly accessible from outside the chip, for example, through a hole extending from fluidic communication with a fluid network to an external surface of the chip, such as the top surface. The receptacles may have a fluid capacity that is relatively large compared to the fluid capacity of the fluid network, so that they are not quickly exhausted. For example, the fluid capacity may be at least about 1, 5, 10, 25, 50, or 100 μL. Accordingly, materials may be dispensed into the receptacles using standard laboratory equipment, if desired, such as micropipettes, syringes, and the like.

Input mechanisms that input materials from external sources also may use any suitable receptacle and mechanism to store and dispense the materials. However, if the external sources input materials directly into the fluid network, the external sources may need to interface effectively with the fluid network, for example, using contact and/or noncontact dispensing mechanisms. Accordingly, input mechanisms from external sources may use capillaries or needles to direct fluid precisely into the fluid network. Alternatively, or in addition, input mechanisms from external sources may use a noncontact dispensing mechanism, such as “spitting,” which may be comparable to the action of an inkjet printer. Furthermore, input mechanisms from external sources may use ballistic propulsion of particles, for example, as mediated by a gene gun.

Facilitating Mechanisms.

The inputting of materials into the microfluidics system may be facilitated and/or regulated using any suitable facilitating mechanism. Such facilitating mechanisms may include gravity flow, for example, when an input reservoir has greater height of fluid than an output reservoir. Facilitating mechanisms also may include positive pressure to push materials into the fluidic network, such as mechanical or gas pressure, or centrifugal force; negative pressure at an output mechanism to draw fluid toward the output mechanism; and/or a positioning mechanism acting within the fluid network. The positioning mechanism may include a pump and/or an electrokinetic mechanism. Positioning mechanisms are further described below. In some embodiments, the facilitating mechanism may include a suspension mechanism to maintain particles such as cells in suspension prior to inputting.

Manufacturing an Instrument Herein.

A mold is fabricated using plural layers of photoresist that are each individually patterned, selectively removed according to the pattern, and optionally rounded by heating. Thus, each of the plural layers may contribute only a subset of the resulting mold, so that the mold's relief pattern is the sum of the remaining portions from each of the plural layers. Using the mold to form a microfluidic network allows various types of channels or other flow channels to be formed. Channels with a rounded/arcuate cross-sectional shape may be formed in sections of the network where valves are needed. These sections may be connected with other portions of the network that are formed to have a rectangular profile, to promote particle movement and to enable precise delivery of one or more particles to a specific area of a microfluidic network. The specific area can be as small as the dimension of a single particle, such as a cell. These structures and other suitable microfluidic structures may be produced using the method described below. This method focuses on formation of a fluid layer, but may be suitable for any portion(s) of a microfluidic system, including a control layer or a base layer.

A fluid-layer mold is fabricated in a first series of steps by micromachining techniques. The fluid-layer mold may be used subsequently in a second series of steps, as described below, to mold a complementary microfluidic layer by soft lithography. A fluid-layer mold may be formed by sequentially disposing, patterning, and selectively removing three layers of photoresist on or above a silicon wafer. Each layer is formed at a desired thickness by applying the photoresist, and then rotating the wafer according to a defined rotational profile to produce the structure. Next, the photoresist is baked, patterned by exposure to UV light, and then developed to selectively remove portions of each layer. To mold closable channels, a photoresist layer may be baked at high temperature to round remaining portions. Each individual step is detailed further below.

The first layer may be applied directly to a bare silicon wafer (the substrate). The first layer may have any suitable thickness, in this case 5 μm, and may be formed with any suitable material, such as a negative photoresist, SU8 2005 (Microchem, Newton, Mass.). After application of the negative photoresist, the wafer may be rotated according to a suitable rotational protocol to achieve a desired thickness and consistency. For example, the wafer may be rotated as follows: rotate to 500 rpm over 5 sec, maintain at 500 rpm for 5 sec, ramp to 3000 rpm over 8 sec, and then maintain at this speed for 30 sec. Then the rotation may be halted and the wafer heated according to a suitable heating protocol. For example, the wafer may be heated for 1 min at 65° C., 2 min at 95° C., and finally 30 sec at 65° C. This heating process may drive off the solvent in which the photoresist may be supplied. The first layer may be patterned and selectively removed as follows. A desired template may be positioned in contact with the first layer and then exposed to UV light, 160 J/cm². Next, the substrate/first layer may be subjected to a suitable post-exposure heating protocol, such as: 1 min at 65° C., 2 min 30 sec at 95° C., and 30 sec at 65° C. Unpolymerized (unexposed) first layer may be washed away with any suitable developer, such as that supplied by Microchem, followed by washing with acetone and then isopropanol. Then, the first layer may be subjected to a suitable post-development heating protocol, such as 1 min at 65° C., 5 min at 95° C., and then 30 sec at 65° C. This heating protocol may be followed by a post-development exposure with UV light, 400 J/cm². A mold with a first layer contributing first-layer relief-structure (residual first layer), which may have a height of 5 μm.

The second layer may be added next and may have any suitable thickness, in this case a thickness of 20 μm formed by spin coating. First, mold may be treated with hexamethyldisilazane (HMDS) for 10 min. Next, a suitable patternable material, such as a positive photoresist, PLP 100 (AZ Electronic Materials/Clariant Corporation) may be applied. Application may be by spin coating, using any suitable protocol, such as the following: spin the wafer at 500 rpm, dispense the positive photoresist to the wafer/residual first layer over 14 sec, spin 15 sec, ramp to 2000 rpm over 5 sec, and maintain at this speed for 30 sec. Rotation then may be stopped, and the second layer may be baked for 2 min at 100° C. The mold, at this intermediate stage, carrying second layer, which covers first-layer relief-structure.

The second layer may be patterned and selectively removed as follows. Any suitable template may be positioned in contact with the second layer and exposed to UV light, 450 J/cm². Next, the second layer may be developed (selectively removed) by any suitable protocol, such as 3 min. in AZ 400K ⅓ with deionized water. The mold has the patterned removal of both first and second layers. First-layer relief-structure and a second-layer relief-structure may have distinct heights based on the thickness of photoresist from which they are formed.

Second-layer relief-structure may be rounded by any suitable heating protocol. For example the structure may be rounded by the following heating protocol: ramp from 70° C. to 100° C. (1° C./min), maintain 60 min at 100° C., ramp to 200° C. (1° C./min), maintain 60 min at 200° C., and ramp down to 40° C. (1° C./min). This heating protocol may convert rectangular second-layer relief-structure to rounded second-layer relief-structure.

A third layer may be added next and may have any suitable thickness, for example, a thickness of 20 μm. A suitable selectively removable material, such as negative photoresist SU8 2050 (Microchem), may be applied to the wafer carrying the residual first and second layers. Spin coating may be achieved by the following protocol: the wafer is ramped to 500 rpm over 5 sec, maintained at this speed for 5 sec, ramped to 5000 rpm over 17 sec, and maintained at this higher speed for 30 sec. The rotation is stopped. Next, the third layer may be heated by any suitable, such as: 2 min. at 65° C., 3 min. at 95° C., and 30 sec at 65° C. A third layer, which covers first-layer and second-layer relief-structures is made at this stage.

The third layer may be patterned and selectively removed as follows. A desired template may be positioned in contact with the third layer and exposed to UV light, 310 J/cm². The exposed layer may be heated by any suitable protocol, such as 1 min. at 65° C., 4 min. at 95° C., and 30 sec at 65° C. Next, the third layer may be selectively removed with a suitable developer, such as that of Microchem, and then may be washed with acetone followed by isopropanol. Subsequently, the third layer may be subjected to a suitable post-development heating protocol, such as 1 min. at 65° C., 5 min. at 95° C., and 30 sec at 65° C. Finally, the third layer may be exposed to UV light in a post-development exposure of 500 J/cm². The mold has a third-layer relief-structure.

Any suitable aspects of the method described above may be modified, and any patternable, selectively removable material may be used. In addition, any suitable number of layers may be used. Furthermore, each layer may have any desired thickness, according to the height of a desired relief structure. When optically patternable layers are used, each layer may be negative or positive photoresist, and may be used to form a rectangular or rounded cross-sectional profile. Relief structures formed by distinct layers may be nonoverlapping, partially overlapping, and/or completely overlapping in specific regions or all regions of the mold. Accordingly, relief structures may represent the sum of plural selectively removed layers.

An exemplary method for forming a control-layer mold is as follows. The mold may be fabricated from a single layer of positive photoresist. A 20-μm layer of suitable photoresist, such as positive photoresist PLP 100, may be applied, patterned, selectively removed, and rounded as described above for the second layer of the fluid-layer mold.

The fluid-layer and control-layer molds fabricated above may be used to mold a microfluidic chip using any suitable material, particularly an elastomeric material, such as polydimethylsiloxane (PDMS). Exemplary PDMS elastomers are General Electric Silicones RTV 615, produced from a two-component mixture of a prepolymer/catalyst and a crosslinker. In this two-component mixture, the prepolymer/catalyst (component A) is a polydimethylsiloxane bearing vinyl groups and a platinum catalyst, and the crosslinker (component B) bears silicon hydride (Si—H) groups. Using these specific components, components A and B may function optimally at a ratio of about 10:1 (A:B). However, “offratios” above and below this ratio may be used for the fluid-layer membrane and the control layer to promote subsequent bonding. For example, the control layer may be formed at a ratio of about 4:1, to provide rigidity and thus mechanical stability, and the fluid-layer membrane at a ratio of about 30:1. The excess of either component A or B in these two layers remain reactive near the membrane surface. Accordingly, these two layers may be abutted and bonded by post-curing with baking to fuse these layers into a monolithic structure.

The fluid-layer and control-layer molds may be fabricated and joined as follows. After treatment with trichloromethylsilane (TCMS), a relatively thin PDMS membrane, for example, about 50-150 μm, may be spun on completed fluid-layer mold. A membrane is formed on fluid-layer mold. In addition, a thicker PDMS layer, for example, approximately 5-10 mm, may be formed on the control-layer mold. After suitable first-step curing, such as 90 min at 80° C., the control layer may be detached from the mold, cut, and punched to interface properly with control lines of the control layer. Then, this control layer may be aligned with the fluid layer, while the fluid-layer membrane is still attached to the fluid-layer mold. Once assembled, the fluid and control layers may be cured a second time to chemically bond them, using a post-curing step of heating for about 3 hours at 80° C. After post-curing, the resulting chip may be detached from the fluid-layer mold, cut, and punched to create fluid reservoirs that interface at desired positions with channels. Finally, the chip may be bonded to a suitable substrate, such as a glass cover slip, to complete the fluid channels.

The post-curing step may be modified to enhance compatibility with cells. Lower ratios of PDMS components A and B, such as 4:1 (A:B), tend to be toxic to cells, particularly during cell culture. This toxicity may be due to a diffusible, toxic material(s) in the control layer. Thus, when a much thicker control layer, formed at a ratio of 4:1, is fused to a thin fluid-layer membrane, formed at a ratio of 30:1, the resulting monolithic structure may have the toxic characteristics of a 4:1 layer, even within the fluid-layer portion. However, suitable treatment of the control layer, either alone in contact with the fluid layer membrane, reduces or eliminates this toxic characteristic. Suitable treatments that remove or modify the toxic material may include exposure to heat, a chemical (such as a gas, a liquid, a plasma, etc.), radiation, light, and/or the like. (Such treatments also may reduce the movement of fluids within the channel, or components thereof, into the chip.) In some embodiments, longer post curing at elevated temperature may remove or modify the toxic material(s), enhancing the effectiveness of the resulting chips for cell experiments. Such a longer post-curing step may be conducted for about 6 hours, 12 hours, or more preferably about 24 hours or more at about 80° C.

Cell Culture and Microfluidic Chip Use.

The CD4 molecule recognizes an antigen that interacts with class II molecules of the major histocompatibility complex (MHC) and is the primary receptor for the human immunodeficiency virus (HIV)(Dalgleish et al., 1984; Maddon et al., 1986). The cytoplasmic portion of the antigen is associated with the protein tyrosine kinase p56kk (Rudd et al., 1989). The CD4 antigen may regulate the function of the CD3 antigen/T-cell antigen receptor (TCR) complex (Kurrle et al., 1989). The CD4 antibody reacts with monocytes/macrophages that have an antigen density lower than that on helper/inducer T lymphocytes (Wood et al., 1983).

The CD8 antigen is present on the human suppressor/cytotoxic T-lymphocyte subset (Evans, et al., 1981; Ledbetter et al., 1981) as well as on a subset of natural killer (NK) lymphocytes (Lanier et al., 1983). The CD8 antigenic determinant interacts with class I MHC molecules, resulting in increased adhesion between the CD8+T lymphocytes and the target cells (Anderson et al., 1987; Eichmann et al., 1987; Gallagher et al., 1988). Binding of the CD8 antigen to class I MHC molecules enhances the activation of resting T lymphocytes. CD8 recognizes an antigen expressed on the 32-kDa α-subunit of a disulfide-linked bimolecular complex (Moebius, 1989). The cytoplasmic domain of the α-subunit of the CD8 antigen is associated with the protein tyrosine kinase p56kk (Rudd et al., 1989; Gallagher et al., 1989).

Determining the percentages of CD4+ and CD8+ lymphocytes may be useful in monitoring the immune status of patients with immune deficiency diseases, autoimmune diseases, or immune reactions. The relative percentage of the CD4+ subset is depressed and the relative percentage of the CD8+ subset is elevated in many patients with congenital or acquired immune deficiencies such as severe combined immunodeficiency (SCID) and acquired immunodeficiency syndrome (AIDS) (Schmidt, 1989; Giorgi, 1990).

The percentage of suppressor/cytotoxic lymphocytes can be outside the normal reference range in some autoimmune diseases (Antel et al., 1986) and in certain immune reactions such as acute graft-versus-host disease (GVHD) and transplant rejection (Gratama et al., 1984; Bishop et al., 1986). The relative percentage of the CD8+ lymphocyte population may often be decreased in active systemic lupus erythematosus (SLE) but can also be increased in SLE patients undergoing steroid therapy (Wolde-Mariam et al., 1984).

The CD4+/CD8+ (helper/suppressor) lymphocyte ratio, quantified as the ratio of CD4 fluorescein isothiocyanate (FITC)-positive lymphocytes to CD8 phycoerythrin (PE)positive lymphocytes, has been used to evaluate the immune status of patients with, or suspected of developing, autoimmune disorders or immune deficiencies (Antel et al., 1986; Wolde-Mariam et al., 1984; Smolen et al., 1982). In many cases, the relative percentages of helper lymphocytes decline and suppressor lymphocytes increase in immune deficiency states. These states may also be marked by T-cell lymphopenia (Ohno et al., 1988). In addition, the ratio has been used to monitor bone marrow transplant patients for onset of acute GVHD (Gratama et al., 1984).

The Jurkat cell, a human mature leukemic cell line, phenotypically resembles resting human T lymphocytes and has been widely used to study T cell physiology. These cells are round, growing singly or in clumps in suspension. They were established from a human T cell leukemia in the peripheral blood of a 14-year-old boy with acute lymphoblastic leukemia (ALL) at first relapse in 1976. This cell line is also called “JM” (JURKAT and JM are derived from the same patient and are sister clones). Occasionally JM may be a subclone with somewhat divergent features confirmed as human with IEF of AST, LDH, and NP. Jurkat cells have the following general restriction properties: CD2+, CD3+, CD4+, CD5+, CD6+, CD7+, CD8−, CD13−, CD19−, CD34+, TCRalpha/beta+, and TCRgamma/delta−.

Tissue samples from humans can be screened for candidate pathogenomonic markers. The data can be correlated with information in databases, including those with information pertaining to environmental pollutant exposures, history of military service, genealogy, and demographic data.

Suitable protocols for performing some of the assays described in this section are included in Joeseph Sambrook and David Russell, Molecular Cloning: A Laboratory Manual (3rd ed. 2000), which is incorporated herein by reference. The principle and mode of operation of this invention have been explained and illustrated in its preferred embodiment. However, it must be understood that this invention may be practiced otherwise than as specifically explained and illustrated without departing from its spirit or scope.

### REFERENCES

[1] V. V. Abhyankar, M. A. Lokuta, A. Huttenlocher, and D. J. Beebe (2006) Lab-on-a-Chip 6:389-393.
[2] J. O. Winter, C. E. Schmidt (2002) Biomimetic Materials and Design: Biointerfacial Strategies, Tissue Engineering, and Targeted Drug Delivery, Marcel-Dekker. pp. 375-415
[3] T. M. Keenam and A. Folch (2008) Lab on a Chip, 8 34-57.
[4] B. G, Chung, L. A. Flanagan, S. W. Rhee, et al (2005) Lab-on-a-chip 5:401-406.
[5] T. M. Keenam, C-H. Hsu, A. Folch, (2006) Applied Physics Letters, 89, 114103.
[6] F. Lin, W. Saadi, S. W. Rhee, S-J. Wang et al (2004) Lab-on-a-Chip 4:164-167.
[7] S. K. W. Dertinger, D. T. Chieu, N. L. ‘eon and G. M. Whitesides, (2001) Analytical Chemistry, 73, 1240-1246.
[8] N. L. Jeon, H. Baskaran, S. K. W. Dertinger, G. M. Whitesides, et al (2002) 20 826-830.
[9] D. B. Weibel, and G. M. Whitesides (2006) Current Opinion in Chemical Biology, 10: 584-591.
[10] J. T. Smith, J. K. Tomfohr, M. C. Wells, et al (2004) Langmuir, 20, 8279-8286
[11] J. P. Ranieri, R. Bellamkonda, J. Jacob at al (1993) Journal of Biomedical Materials Research 27: 917-925.
Abhyankar V V, Lokuta M A, Huttenlocher A, and Beebe D J, (2006), Characterization of a membrane-based gradient generator for use in cell-signaling studies Lab-on-a-Chip 6:389-393.
Bard, A J and Faulkner, L R, (2001), “Electrochemical Methods,” 2nd Edition, John Wiley and Sons, 2001, pp. 137-156
Chung B G, Flanagan L A, Rhee S W, Schwartz P H, Lee A P, Monuki E S, Jeon N L (2005), Human neural stem cell growth and differentiation in a gradient-generating microfluidic device, Lab-on-a-chip 5:401-406.
Dertinger SKW, Chieu D T, Jeon N L and Whitesides G M, (2001), Generation of gradients having complex shapes using microfluidic networks, Analytical Chemistry, 73, 1240-1246.
Fallon M S, Howell B A, and Chauhan A, (2009), Importance of Taylor dispersion in pharmacokinetic and multiple indicator dilution modelling, Mathematical Medicine And Biology—A Journal Of The Ima, (26:4), 263-296
Flanagan J G, (2006), Neural map specification by gradients, Curr Opin Neurobiol. 16(1), 59-66.
Goodhill G J, and Urbach J S, (1999). Theoretical analysis of gradient detection by growth cones. J. Neurobiol. 41, 230-241.
Haessler U, Kalinin Y, Swartz M A, and Wu M, (2009), An agarose-based microfluidic platform with a gradient buffer for 3D chemotaxis studies, Biomed Microdevices 11:827-835.
Hattori K, Sugiura S, and Kanamori T, (2009), Generation of arbitrary monotonic concentration profiles by a serial dilution microfluidic network composed of microchannels with a high fluidic-resistance ratio, Lab-on-a-Chip 9, 1763-1772
Jan Y N and Jan L Y, (2003), The control of dendrite development, Neuron, 40(2):229-42.
Jeon N L, Baskaran H, Dertinger, S K W, Whitesides, G M, Van de Water L, and Toner M (2002), Neutrophil chemotaxis in linear and complex gradients of interleukin-8 formed in a microfabricated device, NATURE BIOTECHNOLOGY, 20(8), 826-830
Keenan T M and Folch A, (2008), Bimolecular gradients in cell culture systems, Lab on a Chip, 8 34-57
Keenan T M, Hsu C-H, Folch A, (2006) Microfluidic “jets” for generating steady-state gradients of soluble molecules on open surfaces, Applied Physics Letters, 89, 114103
Kim M, Yeon J, and Park J K, (2007), A microfluidic platform for 3-dimensional cell culture and cell-based assays, Biomed. Microdevices 9 (1):25-34.
Kim T, Holtzl W J, Park J, Keasling J D, Maharbiz M M, (2009), Pattern Formation In A Synthetic Microbial Pathway Driven By A Microfluidic Platform, Proceedings of 2009 International Conference on Microtechnologies in Medicine and Biology, Québec City, Canada, April 1-3, 110-111.
Lin, F., Saadi, W., Rhee, S. W., Wang, S-J, Mittal S, and Jeon N L, (2004), Generation of dynamic temporal and spatial concentration gradients using microfluidic devices, Lab-on-a-Chip 4:164-167.
Lou J, Shih C Y, Lee E, (2010), Diffusiophoresis of Concentrated Suspensions of Spherical Particles with Charge-regulated Surface: Polarization Effect with Nonlinear Poisson-Boltzmann Equation, LANGMUIR, (26:1), 47-55.
McLaughlin T and O'Leary D D., (2005) Molecular gradients and development of retinotopic maps, Annu Rev Neurosci. 28:327-55.
Ranieri J P, Bellamkonda R, Jacob J G, Gardella J A, Aebischer P, (1993), Selective Neuronal Cell Attachment To A Covalently Patterned Monoamine On Fluorinated Ethylene-Propylene Films, Journal of Biomedical Materials Research 27: 917-925.
Rosoff W J, Urbach J S, Esrick M A, McAllister R G, Richards L J, and Goodhill G J, (2004), A new chemotaxis assay shows the extreme sensitivity of axons to molecular gradients, Nature Neuroscience 7, 678-682.
Schoen I, Krammer H, Braun D, (2009), Hybridization kinetics is different inside cells, Proceedings Of The National Academy Of Sciences Of The United States Of America, (106:51), 21649-21654
Smith J T, Tomfohr J K, Wells M C, Beebe, Jr., Kepler T B, and Reichert W M, (2004), Measurement of cell migration on surface-bound fibronectin gradients, Langmuir, 20, 8279-8286
Salmon E D, Saxton W M, Leslie R J, Karow M L, and McIntosh J R, (1984), Cytoplasm of Embryonic Cells of a Sea Urchin: Video Image Analysis of Fluorescence Redistribution after Photobleaching, The Journal Of Cell Biology, 99, 2157-2164
Smith R L, Burgess R W and Collins, S D, (2009), Combinatorial Cell Microenvironment Generator, Proceedings of 2009 International Conference on Microtechnologies in Medicine and Biology, Québec City, Canada, April 1-3.
Walker G M, Sai J, Richmond A, Stremler M, Chung C Y and Wikswo J P, (2005). Effects of flow and diffusion on chemotaxis studies in a microfabricated gradient generator Lab-on-a-chip 5:611-618.
Weibel D B and Whitesides G M., (2006), Applications of microfluidics in chemical biology, Current Opinion in Chemical Biology, 10: 584-591.
Winter J O, Schmidt C E, (2002), Biomimetic strategies and applications in the nervous system. In: Dillow, A., Lowman, A., ed. Biomimetic Materials and Design: Biointerfacial Strategies, Tissue Engineering, and Targeted Drug Delivery, Marcel-Dekker. pp. 375-415.

### Examples

### Example 1

### Microfluidic Device Materials and Methods Used

A. Optical Measurements

Optical measurements were performed using a Zeiss Axioplan 2 reflection microscope with fluorescence capabilities. Optical visualization of the diffusion profiles were demonstrated using simple organic dyes mixed with water to an appropriate absorbance and used directly. Fluorescence measurements were performed using fluorescein conjugated Bovine Serum Albumin, (BSA) from Molecular Probes with 6.2 fluoresceins per BSA molecule. The BSA was dissolved in phosphate buffered saline (PBS) to a concentration of 0.4 mg/ml and introduced directly in the fluidic microchannels. Agarose (Bacto-Agar, Difco Labs, 0.5% to 1% in DI water) or Matrigel (BD Bioscience, Reduced Growth Factor, LDEV-free) was used as the growth/diffusion medium for the microsystem and test devices.

B. Computer Simulations

Concentration profiles were calculated by finite element volume analysis on an L×L×T (x,y,z) rectangular lattice with lattice spacing Δx=Δy=Δz. based on Equation 1. L was usually normalized to 100 lattice units for all simulations while T, the diffusion medium thickness, varied depending on the particular simulation. Δl is the distance between diffusion ports. Boundary conditions, i.e. sources and sinks, for diffusion ports were set on the xy plane at z=0, Cᵢ(x,y,0,t), according to the conditions given by the particular simulation. Zero flux boundary conditions were imposed at all lattice edges. Steady-state concentrations were generally assumed when concentrations changed less than 1×10⁻⁴ percent over 100 time iterations, although for some experiments the simulations were run considerably longer to verify a steady-state profile. Time was normalized to Δl²/Dᵢ where Dᵢ is the diffusion constant, i.e. 1 second=Δl²/Dᵢ.

C. Microdevice Fabrication

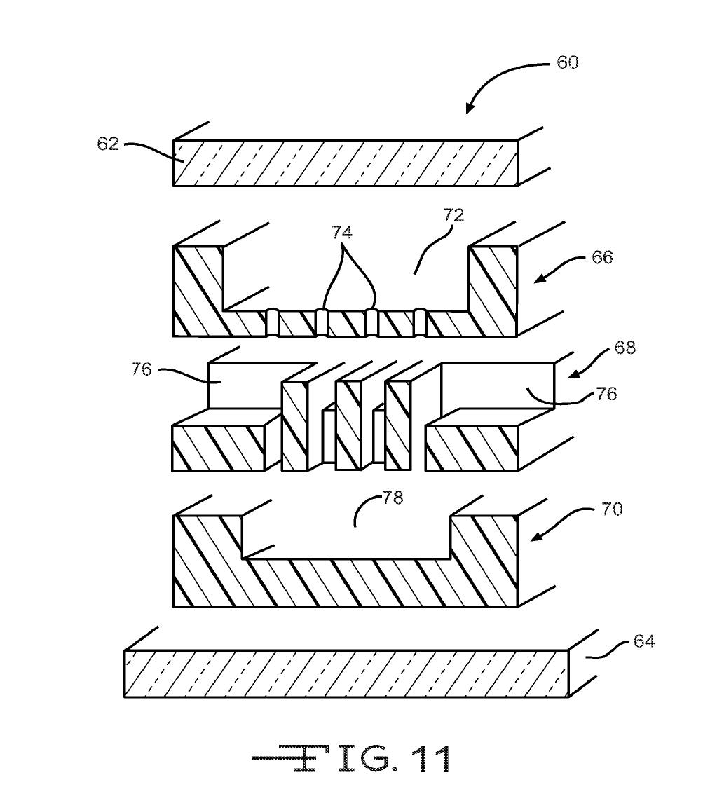

The microfluidic device 60 was fabricated from a multi-layer glass and polydimethylsiloxane (PDMS, Dow Corning, Sylgard 184) stack as shown in FIG. 11. The microfluidic device 60 includes a top glass substrate 62, bottom glass substrate 64, upper core layer 66, intermediate core layer 68 and lower core layer 70. Each PDMS layer was molded against a two level Deep Reactive Ion Etched (DRIE) silicon wafer. The upper core layer 66 consists of the cell culture chamber 72 with the array of diffusion ports 74. The intermediate core layer 68 contains the fluidic microchannels 76 that deliver reagents to the diffusion ports 74, as well as vias to the waste channel. The lower core layer 70 contains the waste channel 78 which can be created by bonding PDMS spacer strips.

PDMS molding was simple and straight-forward. Sylgard 184 was mixed in a 10:1 w/w ratio with its curing agent, degassed in a vacuum, poured into the silicon molds, and then released after curing at 80 C for 2-3 hours. All PDMS layers were aligned and bonded after pre-treating with a one minute oxygen plasma at 250 W, 80 mTorr. Matching keys in each PDMS layer facilitated alignment. After fabrication, the device was typically bonded to a glass substrate for support and ease of handling. Fluidic I/O was provided with 50 μm ID×220 μm OD fused silica microcapillary tubes (Western Analytical Products, Inc.) and standard HPLC fittings and valves. Design of the fluidic microchannels provided a PDMS membrane septum to seal the microcapillary tubes upon insertion into the microfluidic channels.

The microdevice was prepared for experiments by addition of a culture medium in the culture chamber (agarose or Matrigel), and a removable glass cover slip was placed over the culture chamber to facilitate microscopy observation and reduce evaporation. All measurements were performed at room temperature under ambient conditions.

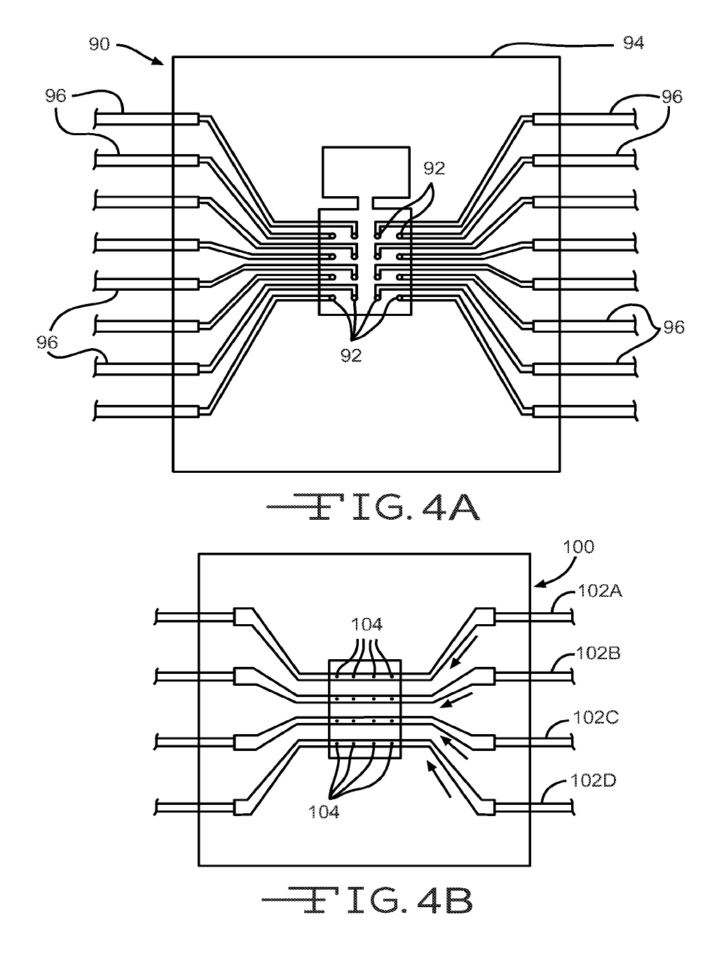

FIG. 4(a) illustrates a microfluidic device 90 with a 4×4 array of 20 μm×20 μm diffusion ports 92. The outline of the cell growth chamber 94 is seen as the large rectangle on the top layer. The diffusion ports 92 are visible at the end of each fluidic microchannel 96. Fluidic vias to the waste channel lie immediately below the end of the microchannels 96, but are not shown. Shown in FIG. 4(b) is a modified embodiment of a similar microfluidic device 100 with a reduced number of fluidic I/O connections. This design is useful for many simple diffusion profiles. Here four straight microchannels 102a, 102b, 102c, and 102d address a series of eight diffusion access ports 104 distributed along the length of each channel. For ease of illustration only 4 access ports 104 are shown in each flow channel in FIG. 4(b).

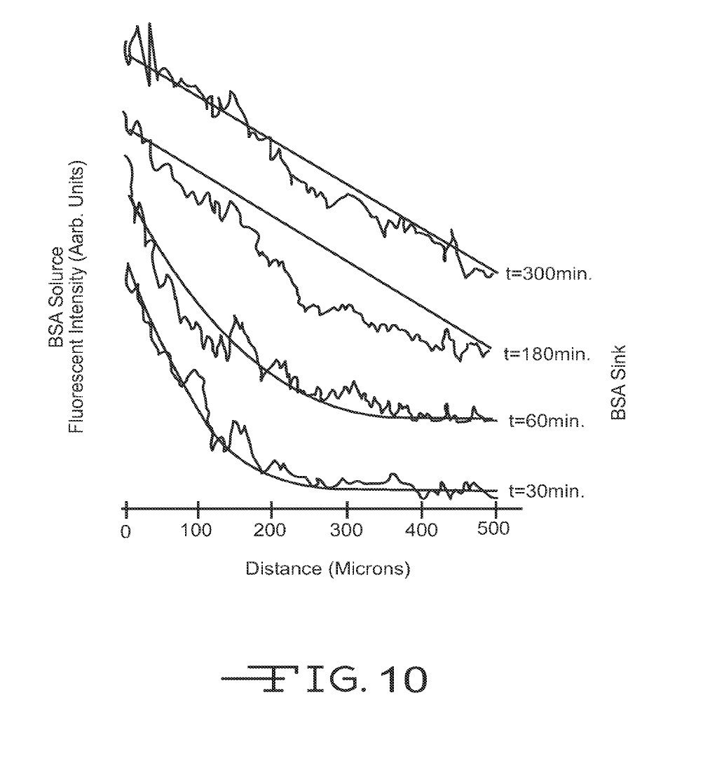

The cell culture chamber for the above-described microfluidic devices was typically 1-2 mm×3-4 mm with depths ranging anywhere between 30 and 200 μm depending on the particular microfluidic device design and intended application. For most device embodiments spacing between the diffusion ports, Δl, varied between 300 and 400 μm unless otherwise noted. The diffusion port openings ranged from 4 μm×4 μm to 20 μm×20 μm, and the fluidic microchannels were typically 100 μm×200 μm in cross sectional area. After introducing liquid into the microchannels, agarose or Matrigel was pipetted into the culture chamber and capped with a standard microscope cover slip. Using this procedure no bubble formation or microchannel filling difficulties were observed. Although biological cells were not used in this study, the design of the chamber provides for either imbedding the cells within the growth matrix or plating cells on the surface of the matrix using standard protocols developed for the specific cell type used. Fluid to the microchannels was supplied continuously either by gravity feed from an elevated reservoir or from a pressurized reagent bottle (1-10 psi) and regulated with a needle valve. Both methods provided consistent, low pressure, and pulse-less flows. Flow through the microchannels is required to maintain the concentration at the diffusion ports constant. Although this flow rate depends somewhat on the magnitude of the reagent diffusion constants, it can be easily estimated by assuming that the fluid across an access port must be replenished completely every second. With the dimensions of this device, this results in typical flow rates of <10 pL/min per microchannel which has been confirmed experimentally under actual diffusion testing of proteins (see below, FIG. 10). Small fluid volumes are important when studying biological reagents that are rare and/or expensive.

### Example 2

### Microfluidic Device Results

A. Computer Simulations

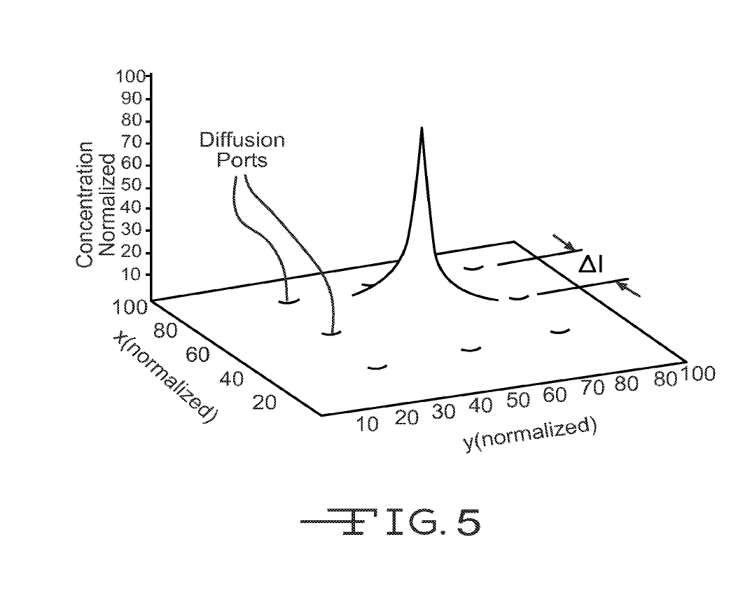

Diffusion is the driving force behind the formation of concentration profiles, and the fundamental equations driving diffusion are Fick's first and second laws.

$$\begin{matrix}{J}_{i} = - {D}_{i} ∇ {C}_{i} & Eq 1 \\ \frac{\partial {C}_{i} ( x , y , z , t )}{\partial t} = - {D}_{i} {∇}^{2} {C}_{i} ( x , y , z , t ) & Eq 2\end{matrix}$$

Where Jᵢ is the flux and Dᵢ is the diffusion coefficient or diffusivity of species i, Cᵢ(x,y,z,t) is the concentration of i at point (x,y,z,t) and ∇□ is the del operator (Bard, 2001). A finite element simulation, FIG. 5, for a 5×5 array of diffusion ports spaced 25 lattice units apart, Δl=25, was performed with the center diffusion port as a single concentration source with a normalized concentration of 100, Cᵢ(50,50,0,t)=100, and the remaining diffusion ports held at zero. FIG. 5 shows the resulting, steady-state, 2D concentration profile on the xy plane at z=0, i.e. the concentration profile at (x,y,0,t=∞). The source diffusion port is clearly seen as the peak at the center of the field, while the sink diffusion ports are seen as an array of depressions in the concentration field surrounding the center peak. This profile demonstrates the base Laplacian profile that is obtained for each individual diffusion port. More complex profiles are obtained through combinations of this basic profile, as described below.

In FIG. 5 there is illustrated a hypothetical diffusion profile indicating the diffusion into the culture chamber from multiple access ports fed by supply channels having various concentrations of agents.

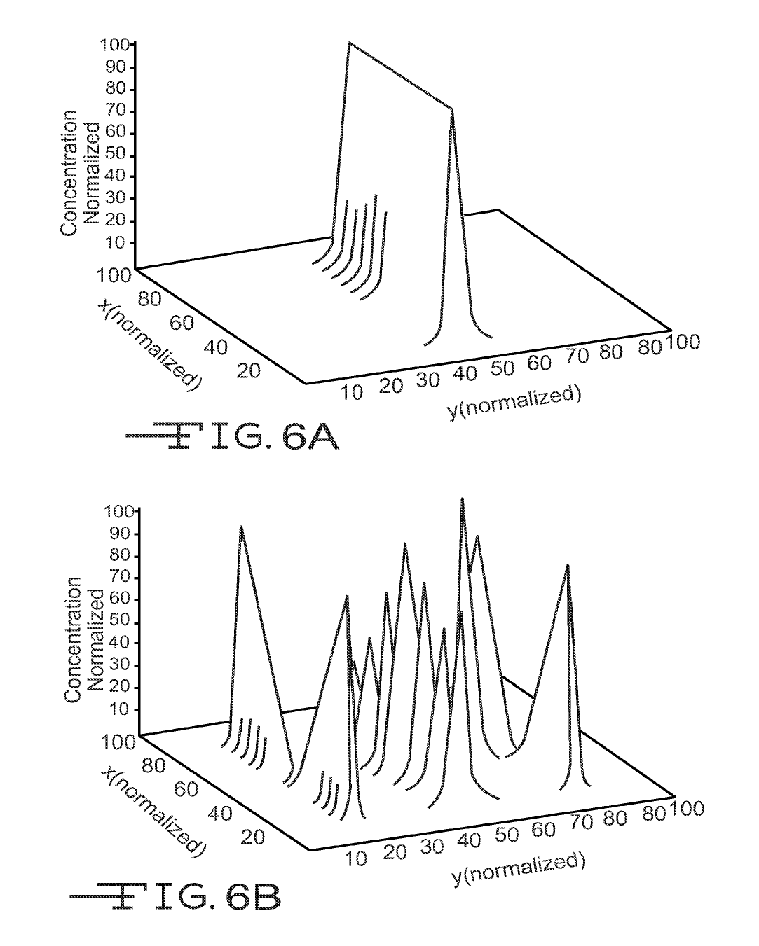

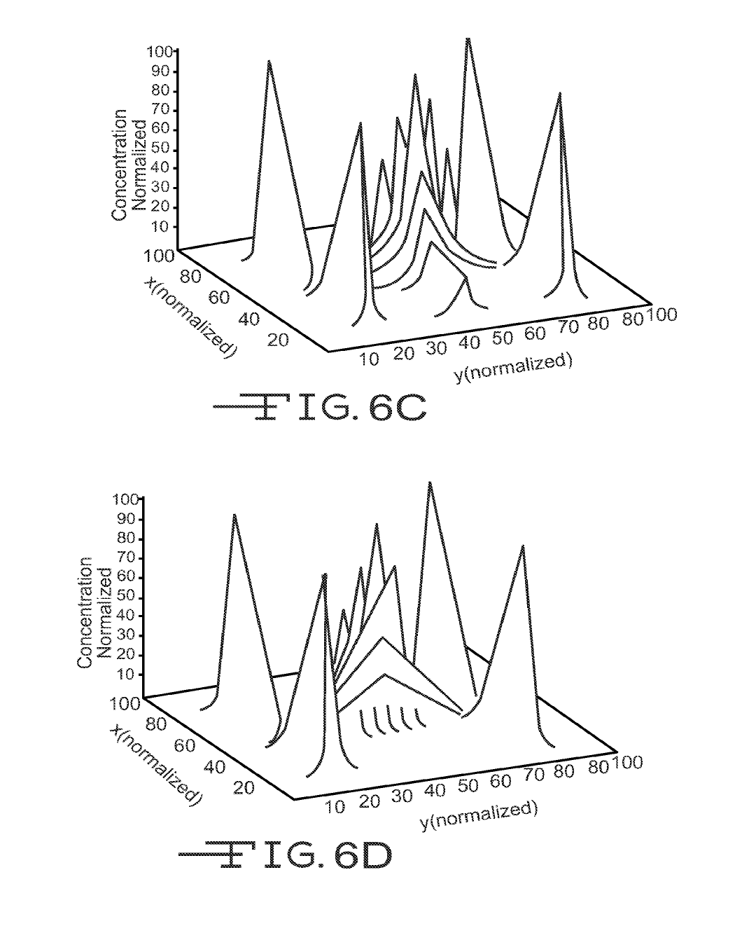

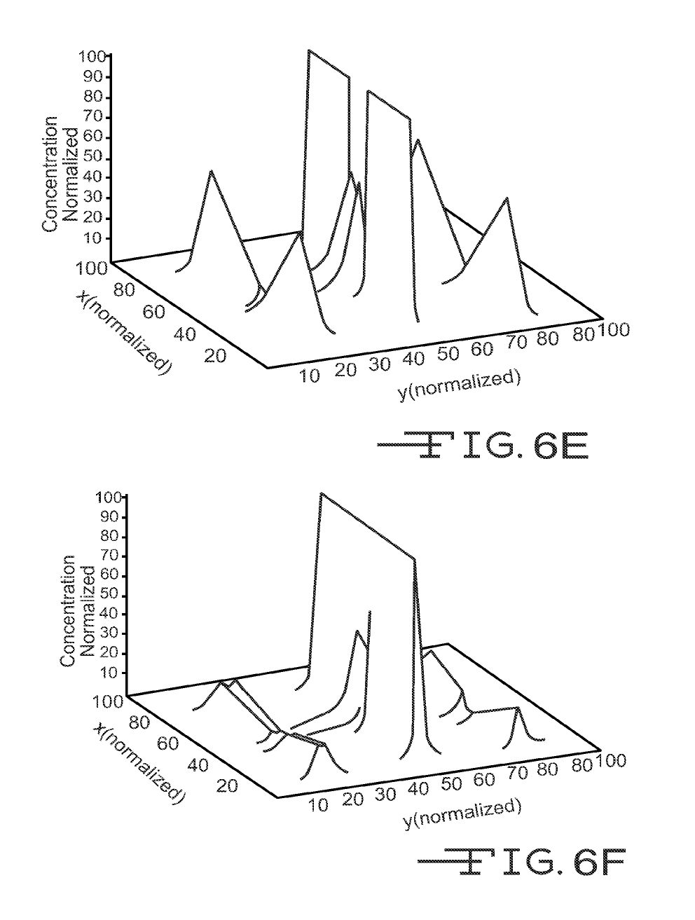

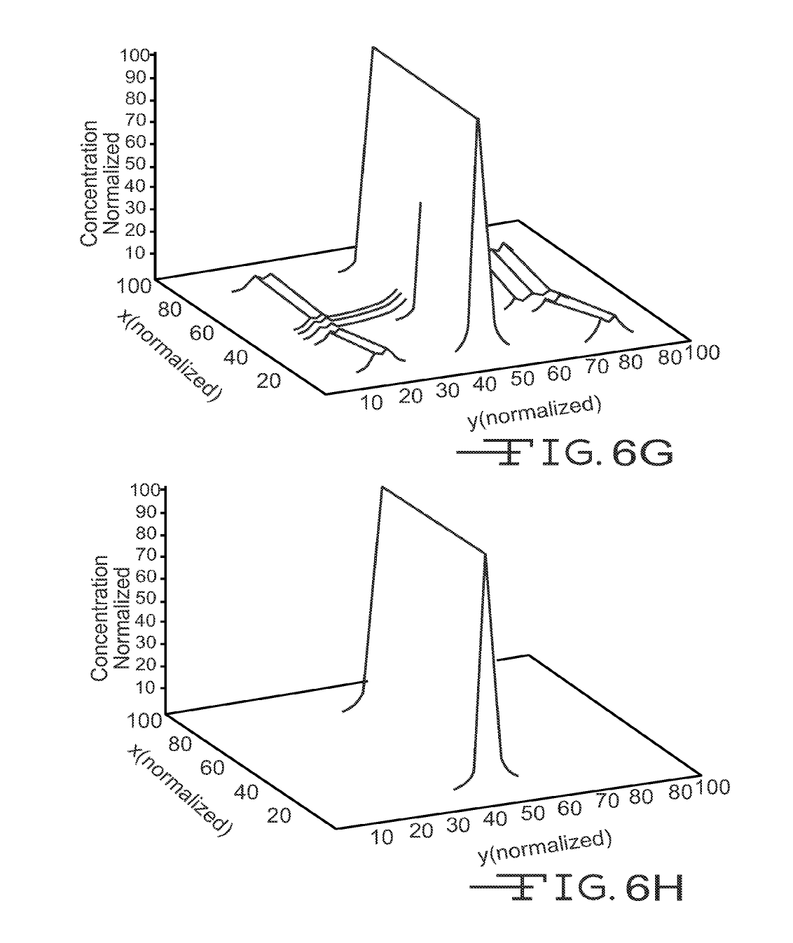

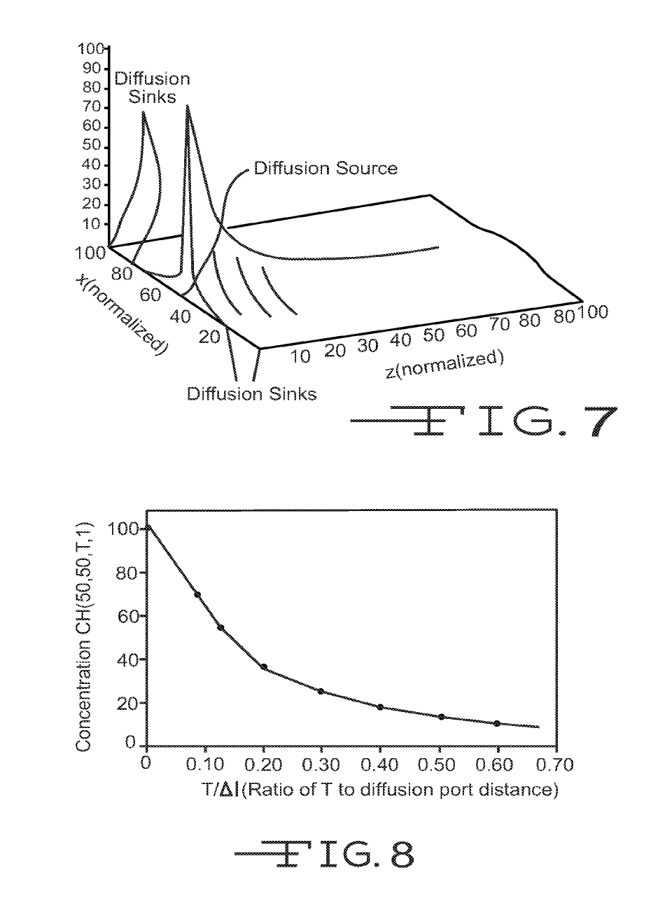

FIG. 6 shows a series simulations for a 19×19 array of diffusion ports, Δl=5 lattice units, that are individually addressed with varying concentrations in both space and time. The first simulation, FIG. 6(a), shows the 2D, steady-state diffusion profile, Cᵢ(x,y,0,t=∞), obtained by addressing a linear series of 13 diffusion ports with a normalized source concentration of Csource=100 and the remaining diffusion ports in the array with zero concentration, Csink=0. At a specific time, t_{zero}, the concentrations of all the diffusion ports are changed, and the transition to a new steady state concentration profile is shown in the progressive time series, 6(b)-6(d). FIG. 6(d) is the final steady-state concentration profile obtained after t_{zero}+40 sec (normalized), and the reagent concentration at each diffusion port can be easily determined by the peak concentration value at each diffusion port. At t_{zero}+110 sec the concentrations at each diffusion port were again switched back to their original concentrations and FIGS. 6(e)-6(h) show the transition back to the original steady-state, linear diffusion of FIG. 6(a).

Concentration profiles extend not only in the xy plane as depicted in FIGS. 5 and 6, but three dimensionally along the z axis as well. FIG. 7 depicts a typical steady state concentration profile in the xz plane for the same simulation conditions as used in FIG. 5, i.e. a single source, Cᵢ(50,50,0,t)=100. The xz plane is centered on the source diffusion port, (x,50,z, t=∞). The thickness of the lattice in the z direction is 15 lattice units, T=15. As expected, the concentration tails off monotonically in the z direction and becoming more diffuse in the xy plane with increasing distance from the source diffusion port. However, the concentration profile in the z direction depends not only on the thickness of the diffusion medium, T, but also on the distance between the diffusion ports in the xy plane as well. FIG. 8 plots the concentration peak at T, i.e. Cᵢ(50,50,T, t=∞) as the ratio of T/Δl, where Δl varies from 10-50 lattice units and T varies from 3 to 20 lattice units. As the diffusion medium thickness, T, approaches the spacing between the diffusion ports, T/Δl→1, the concentrations along the z axis approach a small and uniform profile in the xy plane typical of planar diffusion and shown in FIG. 7. However, as T/Δl decreases, the concentrations in the xy plane at T approach the values for 2D diffusion on the xy plane at z=0.

Concentration profiles typified by large T/Δl are probably more representative of that which occurs naturally in vivo where the spatial extent of the diffusion medium can be extensive while the sources and sinks can be quite dense. However, in many in vitro studies it is sometimes advantageous to reduce the system to a 2D problem where the concentration in the z direction is essentially constant. In this case, providing a diffusion medium where T/Δl<0.10 assures that the concentration in the z axis will vary less than 75% from 0<z<T.

Due to ease of presentation, only one diffusing species is applied throughout all the above simulations. However, it is a simple matter to superimpose simultaneous, multiple concentration profiles for any number of different species by supplying the appropriate chemical cocktail to each diffusion port, i.e. one gradient field for pH can be established with an entirely different and independent spatial/temporal field for pO2, glucose, metabolites, mediators, etc. As long as each species behaves independently, i.e. no chemical coupling occurs between the reagents, each diffusion field will develop independently. In this way, complex or opposing gradients in different or similar axes can be established for different chemical species; a situation that more closely approximates the in vivo condition (Flanagan, 2006; McLaughlin, 2005).

### Example 3

### Microfluidic Device Experimental Verification

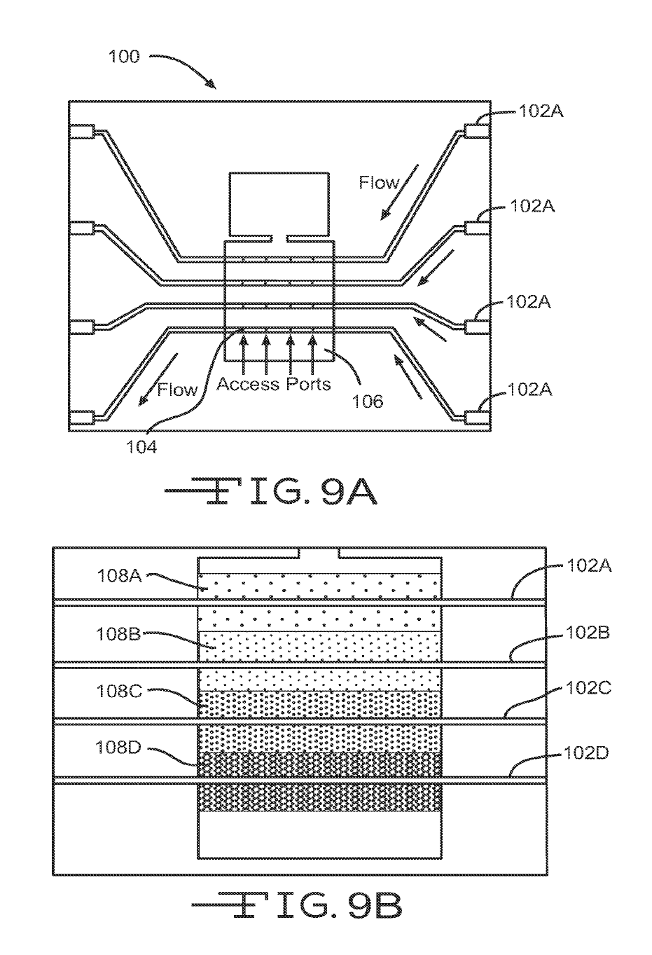

To experimentally validate the results of the diffusion simulations and demonstrate the power and versatility of the diffusion microsystem to control both temporal and spatial diffusion profiles, the device shown in FIG. 4(b) was characterized experimentally by addressing each microfluidic channel with a differently colored organic dye. There are eight, 20 μm×20 μm diffusion ports 104 spaced equally along the length of each microchannel, although for ease of illustration only 4 are shown in FIG. 4(b). The separation between microchannels is approximately 1 mm. FIG. 9(a) shows the microdevice 100 with a different color dye loaded in each microchannel 102a, 102b, 102c and 102d at the beginning of the experiment before diffusion profiles have become established, i.e. t=0. Channel 102a was filled with blue die, channel 102b was filled with green die, channel 102c was filled with orange die, and channel 102d was filled with red die. FIG. 9(b) shows the same set of channels 102a, 102b, 102c and 102d after the dyes were allowed to diffuse into the culture 106 for 30 minutes, although steady-state profiles were well established in less than 10 minutes. This concentration profile remained constant for the duration of the experiment, in excess of 4 hours, with the exception that the blue and red dyes continually diffused into the upper and lower portions of the chamber where there were no diffusion ports to sink the concentration. As shown in FIG. 9(b), each different color is represented by a different shading to indicate diffusion into the culture 106. As shown, as each die diffused into the culture 106 through the ports 104, a zone of dyed or affected culture was observed, indicated in FIG. 9(b) as zones 108a, 108b, 108c and 108d, respectively for the blue, green, orange and red dyes.

Each set of eight diffusion ports 104 served as the source concentration for the specific dye loaded in that channel 102a, 102b, 102c and 102d while the remaining diffusion ports 104 served as the concentration sinks for that dye. Since each dye is a distinct chemical entity and does not chemically react with the other dyes, they all diffuse independently and set up four parallel concentration profiles each one similar to those demonstrated in the computer simulations of FIGS. 6(a, h), but offset spatially over each microchannel. The overall diffusion field of the culture chamber is just the summation of the four independent diffusion fields. This allows the user the freedom to create complex, steady state diffusion fields with multiple species having similar or opposing gradients, dynamically changing gradients, etc. At the end of the experiment, all microfluidic channels 102a, 102b, 102c and 102d were switched to distilled water and within 40 minutes all visible traces of the dyes disappeared from the interior chamber indicating that concentration profiles can be dynamically and reversibly changed over the course of an experiment.

Table 1 lists the calculated times required to reach steady-state concentration profiles as a function of both the diffusion constant and the distance between access ports as per Equations 1 and 2. Since the colored dyes required approximately 10 min to establish a steady-state concentration gradient over a 1 mm distance, it can estimated from Table I that the diffusion constants for the organic dyes are approximately 6×10⁻⁶ cm²/sec which is typical for large organic molecules (Bard, 2001). Proteins typically have diffusion constants <1×10⁻⁷ cm²/s in agarose/Matrigel matrices (Goodhill, 1999) and would require a correspondingly longer time to establish steady-state profiles.

TABLE 1
Time (min.) to Establish a 95% Steady-State Concentration Profile

| | |
|---|---|
| Diffusion | Diffusion Distance, μl (microns) |

| Coeff., D | 100 | 300 | 500 | 700 | 1,000 |
|---|---|---|---|---|---|
| (cm²/sec) | μm | μm | μm | μm | μm |
| 1.0 × 10⁻⁵ | 0.03 | 0.41 | 1.25 | 2.54 | 5.35 |
| 8.0 × 10⁻⁶ | 0.04 | 0.51 | 1.56 | 3.18 | 6.68 |
| 4.0 × 10⁻⁶ | 0.08 | 1.03 | 3.12 | 6.35 | 13.37 |
| 1.0 × 10⁻⁶ | 0.31 | 4.12 | 12.47 | 25.42 | 53.47 |
| 8.0 × 10⁻⁷ | 0.39 | 5.15 | 15.59 | 31.77 | 66.83 |
| 4.0 × 10⁻⁷ | 0.78 | 10.30 | 31.18 | 63.55 | 133.66 |
| 1.0 × 10⁻⁷ | 3.13 | 41.19 | 124.74 | 254.20 | 534.67 |
| 8.0 × 10⁻⁸ | 3.91 | 51.49 | 155.93 | 317.75 | 668.33 |
| 1.0 × 10⁻⁸ | 31.34 | 422.8 | 1247.8 | 2542.0 | 5346.7 |

To quantify the concentration profiles as a function of time, fluorescein conjugated Bovine Serum Albumin was introduced into one microfluidic channel, the source, while the other microchannels received water, the sinks Spacing between the source and sink was 500 In this configuration, diffusion profiles reduce to essentially a one dimensional problem in the x direction as shown in FIGS. 6a and 6h. FIG. 10 shows the developing concentration profiles at t=30 min, 60 min, 180 min, and 300 min. Steady-state profiles developed after about 3.5 hours (210 min) indicating from Table I that BSA has a diffusion coefficient in agarose of approximately 6×10⁻⁸ cm²/s which is close to that found in vivo, 8×10⁻⁸ cm²/s, and lower than that of BSA in water, 1×10⁻⁷ cm²/s (Salmon, 1984).

For short times when t<<Δlᵢ²/2Dᵢ the diffusion profiles develop under essentially semi-infinite diffusion conditions. Solution of Eq 2 under these boundary conditions yields profiles that are governed by

$${C}_{i} ( x , t ) = {C}_{i}^{o} [ erfc ( \frac{x}{2 {( {D}_{i} t )}^{1 / 2}} ) ] \qquad Eq . 3$$

for 1D diffusion (Bard 2001), where C^{o}₁ is the concentration of the i^{th} species at x=0 and t=0, i.e. Cᵢ(0,0). Concentration profiles for t=30 min and t=60 min show semi-infinite diffusion behavior and were iteratively fit to Eq. 3. FIG. 10 (t=30 min and t=60 min) show the experimental and calculated concentration values for the diffusion of BSA. Best fit curves were obtained using a diffusion coefficient of 3×10⁻⁸ cm²/s which is consistent with previously reported values and estimates obtained from Table I. At about 100 min the profiles began to transition into the steady-state linear profiles expected from a 1D diffusion configuration. The profile at t=180 min shows an incomplete transition to linear while the profile at t=300 min shows complete transition and the expected steady-state linear profile.

It should be noted that in this experiment, flow rates in the microchannels were only 10-30 pL/min. which was sufficient to maintain the fluorescein/BSA concentrations at the boundary source and sink diffusion ports constant. Small flow volumes are important when expensive or rare bioreagents are used. In this experiment, less than 0.8×10⁻⁹ g/hr of BSA was used.

### Example 4

### Microfluidic Device and Cell Culture

Cells Used.

LNCaP is an androgen-dependent human prostate cancer cell line with the expression of androgen receptor, PSA, and PSMA. CWR22rv is an androgen-independent prostate cancer cell line derived from an androgen-dependent human xenograft tumor, CWR22s. C4-2, CWR22rv were all maintained in RPMI 1640 supplemented with 10% fetal bovine serum and 1% penicillin/streptomycin.

Cells on PDMS Microfluidic Devices.

A microfluidic device was attached on the bottom of one well of 6-well plates, and triplicates were used. 1×10⁵ LNCaP cells were seeded on the microfluidic device in 500 μL culture media. The microfluidic device and cells were monitored under the light microscope at five minutes. The cells were evenly located in the culture chamber.

Cell Growth on Collagen/No Plasma, Collagen/Plasma Microfluidic Devices.

Three types of microfluidic devices having one of three media types, including PDMS, collagen/no plasma and collagen/plasma, were each attached on the bottom of one well of 6-well plates, and triplicates were used for each chip. 1×10⁶ CWR22rv cells were seeded in 1 mL culture media. The cells were cultured for 24 hours at 5% CO2 and 37° C. incubator. The microfluidic device and cells were observed under the light microscope at 24 hours after cell seeding. The cells on the microfluidic devices with collagen plus plasma grew faster than the cells on microfluidic devices with collagen/no plasma, or PDMS.

1-168. (canceled)

168. A microfluidic device comprising
at least one cell culture chamber configured to receive and retain media;
at least three diffusion ports in fluid communication with said cell culture chamber;
at least three flow channels functionally connected to said diffusion ports;
at least one regulator functionally connected to said flow channels and said diffusion ports to regulate the circulation of at least one material into said flow channels and said diffusion ports to affect a concentration gradient of said at least one material in said cell culture chamber to evaluate the effect of said at least one material on a cell culture in said cell culture chamber,
wherein at least one said diffusion port acts as an inlet port for said at least one material and at least one diffusion port acts as an outlet port for said at least one material.

169. The microfluidic device of claim 168, wherein said at least one material is circulated via passive diffusion.

170. The microfluidic device of claim 168, wherein the device further comprises one or more waste channels that are in fluid communication with said cell culture chamber.

171. The microfluidic device of claim 168, wherein the device comprises between 3 and 100 diffusion ports.

172. The microfluidic device of claim 168, wherein the device comprises between 3 and 100 flow channels.

173. The microfluidic device of claim 168, wherein one or more of said diffusion ports does not have a cross sectional dimension that exceeds 130 micrometers.

174. The microfluidic device of claim 168, wherein one or more of said diffusion ports has a largest cross sectional dimension that is between about 80 micrometers and about 100 micrometers.

175. The microfluidic device of claim 168, wherein said chamber substantially comprises an elastomer, a plastic, a ceramic, glass, a photoresist, a metalloid, a metal or metal oxide, a biological polymer, or a combination thereof.

176. A method of manufacturing a microfluidic device, the method comprising
providing a cell culture chamber configured to receive and retain media;
introducing at least three diffusion ports into said cell culture chamber;
connecting at least three flow channels to said diffusion ports;
providing at least one regulator configured to regulate the circulation of at least one material into said flow channels and said diffusion ports to affect a concentration gradient of said at least one material in said cell culture chamber to evaluate the effect of said at least one material on a cell culture in said cell culture chamber; and
connecting said regulator to said flow channels and said diffusion ports, wherein at least one said diffusion port acts as an inlet port for at least one material and at least one diffusion port acts as an outlet port for at least one material.

177. The method of claim 176, wherein said at least one material is circulated via passive diffusion.

178. The method of claim 176, wherein the device further comprises one or more waste channels that are in fluid communication with said cell culture chamber.

179. The method of claim 176, wherein the device comprises between three and 100 diffusion ports.

180. The method of claim 176, wherein the device comprises between 3 and 100 flow channels.

181. The method of claim 176, wherein one or more of said diffusion ports does not have a cross sectional dimension that exceeds 130 micrometers.

182. The method of claim 176, wherein one or more of said diffusion ports has a largest cross sectional dimension that is between about 80 micrometers and about 100 micrometers.

183. The method of claim 176, wherein said chamber substantially comprises an elastomer, a plastic, a ceramic, glass, a photoresist, a metalloid, a metal or metal oxide, a biological polymer, or a combination thereof.

184. A method of establishing a concentration gradient, the method comprising the steps of
providing a microfluidic device comprising
at least one cell culture chamber configured to receive and retain media;
at least three diffusion ports in fluid communication with said cell culture chamber;
at least three flow channels functionally connected to said diffusion ports;
at least one regulator functionally connected to said flow channels and said diffusion ports and configured to regulate the circulation of at least one material into said flow channels and said diffusion ports to affect a concentration gradient of said at least one material in said cell culture chamber to evaluate the effect of said at least one material on a cell culture in said cell culture chamber,
wherein at least one said diffusion port acts as an inlet port for at least one material and at least one diffusion port acts as an outlet port for at least one material;
providing media;
introducing said media to said microfluidic device;
providing at least one material;
circulating said at least one material across at least a portion of said chamber, wherein said at least one material is conducted into said chamber through at least one said flow channel and at least one said diffusion port and then conducted out of said chamber by at least one said flow channel and at least one said diffusion port to form a concentration gradient in the cell culture chamber.

185. The method of claim 184, wherein said at least one material is circulated via passive diffusion.

186. The method of claim 184, wherein the device further comprises one or more waste channels that are in fluid communication with said cell culture chamber.

187. The method of claim 184, wherein the device comprises between three and 100 diffusion ports.

188. The method of claim 184, wherein the device comprises between 3 and 100 flow channels.

189. The method of claim 184, wherein one or more of said diffusion ports does not have a cross sectional dimension that exceeds 130 micrometers.

190. The method of claim 184, wherein one or more of said diffusion ports has a largest cross sectional dimension that is between about 80 micrometers and about 100 micrometers.

191. The method of claim 184, wherein said chamber substantially comprises an elastomer, a plastic, a ceramic, glass, a photoresist, a metalloid, a metal or metal oxide, a biological polymer, or a combination thereof.
